(19) United States
(12) Patent Application Publication
(10) Pub. No.: US 2024/0216439 A1
(43) Pub. Date: Jul. 4, 2024

(54) COMPOSITIONS OF CELLS WITH AN ENHANCED THERAPEUTIC CAPACITY

(71) Applicant: BONUS THERAPEUTICS LTD., Haifa (IL)

(72) Inventors: Tomer BRONSHTEIN, Haifa (IL); Dror BEN DAVID, Kiryat Motzkin (IL); Atara NOVAK, Haifa (IL); Shai MERETZKI, Haifa (IL)

(73) Assignee: BONUS THERAPEUTICS LTD., Haifa (IL)

(21) Appl. No.: 18/563,558

(22) PCT Filed: May 23, 2022
(86) PCT No.: PCT/IL2022/050536

§ 371 Date: Nov. 22, 2023

Related U.S. Application Data
(60) Provisional application No. 63/192,049, filed on May 23, 2021.

(51) Int. Cl. A61K 35/28; A61P 29/00

(52) U.S. Cl. CPC A61K 35/28; A61P 29/00

(57) ABSTRACT

The present invention provides a composition including target mesenchymal cells and methods of using same, such as for inhibiting or controlling immune response, such as inflammation, inducing tissue regeneration, and inducing wound healing in a subject in need thereof.

### CROSS-REFERENCE TO RELATED APPLICATION

This application claims the benefit of priority of U.S. Provisional Patent Application No. 63/192,049, titled “COMPOSITIONS OF CELLS WITH AN ENHANCED THERAPEUTIC CAPACITY”, filed May 23, 2021, the contents of which are incorporated herein by reference in their entirety.

### FIELD OF INVENTION

The present invention is generally in the field of cell biology, and particularly in target mesenchymal cells, such as for use in regenerative medicine and regulation of the immune response.

### BACKGROUND OF THE INVENTION

Mesenchymal stromal cells—are non-hematopoietic, multipotent, self-renewable cells that are capable of trilineage differentiation (mesoderm, ectoderm, and endoderm). The pluripotency, regenerative, and immunomodulatory features of Mesenchymal stromal cells make them an effective tool for cell therapy and tissue repair.

Mesenchymal stromal cells (MSC) have attracted much attention for treating different indications involving tissue degeneration, inflammation, and autoimmunity. This is owing to the anti-inflammatory and immunomodulatory capacity of MSCs and their ability to support tissue regeneration, for which they are already tested in numerous clinical studies. Despite their preclinical success, MSCs' clinical translation is overshadowed by their failure to show meaningful clinical efficacy. By most, this limited efficacy is attributed to the variability between cell batches, lack of standardization in MSC production, culturing, and characterization processes, and the loss of MSC therapeutic potency during their in vitro expansion and storage. Moreover, a major obstacle that may hinder MSCs' clinical translation is the poor survival and viability of MSCs post-transplantation, which is thought to occur as cells are exposed to oxidative stress, loss of matrix attachments, nutrient, and serum deprivation, and in some cases, are placed in relatively hypoxic, ectopic environment to that in which they usually reside (Moloney et al., 2012; Silva et al., 2018). Also, concerns regarding the hemocompatibility of MSCs and the risk of causing adverse thromboembolic events were raised vis-à-vis the cells' source tissue and administration route.

### SUMMARY OF THE INVENTION

According to one aspect, there is provided a composition comprising target mesenchymal cells characterized by overexpression of BCL2 and FGF7 genes compared to expression of the BCL2 and the FGF7 in control mesenchymal cells; and under-expression of HSP90AA1, MTOR, and AKT1 genes compared to expression of the, HSP90AA1, said MTOR and said AKT1 in the control mesenchymal cells.

According to another aspect, there is provided a method for inhibiting an immune response in a subject in need thereof, comprising administering to the subject a therapeutically effective amount of the composition disclosed herein, thereby inhibiting an immune response in a subject in need thereof.

According to another aspect, there is provided a method for inducing tissue regeneration in an injured tissue or a degenerated tissue in a subject in need thereof, comprising administering to the subject a therapeutically effective amount of the composition disclosed herein, thereby inducing tissue regeneration in an injured tissue or a degenerated tissue in a subject in need thereof.

In some embodiments, the target mesenchymal cells are further characterized by under-expression of PAK1 gene compared to expression of said PAK1 gene in the control mesenchymal cells and less than 22% under-expression of THBD gene compared to expression of said THBD gene in the control mesenchymal cells.

In some embodiments, the target mesenchymal cells are further characterized by overexpression of EDIL3 gene, AMOT gene, or their combination, compared to expression of the EDIL3, AMOT, or both in the control mesenchymal cells.

In some embodiments, the target mesenchymal cells are further characterized by overexpression or de novo expression of the IL6R gene compared to expression of the IL6R in the control mesenchymal cells.

In some embodiments, the target mesenchymal cells are further characterized by increased cell granularity compared to control mesenchymal cells, wherein the target mesenchymal cells are further characterized by a larger proportion of cells in the G1 phase of a cell cycle compared to control mesenchymal cells, or a combination thereof.

In some embodiments, the overexpression of BCL2 is by at least 1.5-fold increase, wherein the overexpression of FGF7 is by at least 5-fold increase, wherein the under-expression of HSP90AA1 is by at least 2-fold decrease, wherein the under-expression of MTOR is by at least 1.5-fold decrease, wherein the under-expression of AKT1 is by at least 1.5-fold decrease, wherein the under-expression of PAK1 is by at least 1.4-fold decrease, or any combination thereof.

In some embodiments, the under-expression of PAK1 is by at least a 1.8-fold decrease.

In some embodiments, the overexpression of EDIL3 is by at least 7.3-fold increase, wherein the overexpression of AMOT is by at least 1.5-fold increase, or both.

In some embodiments, the mesenchymal cells are derived from: adipose tissue, umbilical cord, chorionic placenta, bone marrow, amniotic placenta, dental pulp, amniotic fluid, peripheral blood, synovium, synovial fluid, endometrium, skin, muscle, embryonic stem cells, induced pluripotent stem cells, or any combination thereof.

In some embodiments, the target mesenchymal cells are modified by subjecting the cells to a combination of conditions selected from the group consisting of: (a) hypoxia, (b) starvation, (c) oxidative stress, (d) hypothermia, (e) hyperthermia, (f) over confluency, (g) hydrostatic pressure, (h) dynamic or cyclic pressure, (i) shear forces, (j) agitation, (k) exposure to charged surfaces, (l) under confluency; and (m) any combination of (a) to (l).

In some embodiments, the target mesenchymal cells are targeted or suitable for use in inhibition of an immune response in a subject in need thereof.

In some embodiments, the method further comprises reducing inflammation in the subject.

In some embodiments, the immune response is an adaptive immune response, an innate immune response, or both.

In some embodiments, the subject is: afflicted with a cytokine storm, afflicted with a cytokine release syndrome, afflicted with SEPSIS and/or SIRS, at risk of developing a cytokine storm, at risk of developing a cytokine release syndrome, or at risk of developing SEPSIS and/or SIRS.

In some embodiments, the inhibiting comprises inhibiting the activity of CD4+ cells, CD8+ cells, or both.

In some embodiments, the inhibiting comprises inhibiting cytokine production, secretion, or both, in the subject.

In some embodiments, the tissue regeneration comprises induction of wound healing, reendothelization, reepithelization, cell proliferation, cell migration, or any combination thereof.

In some embodiments, the tissue regeneration comprises inhibiting or controlling tissue fibrosis.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1A-1B are graphs showing flow cytometric analysis of two representative batches, i.e., Batch A (1A) and Batch B (1B), of target MSCs and their paired control MSCs revealing a substantial 47% to 52% increase in the geometric mean of the side scatter (SSC) parameter measured for target MSCs, pointing to their increased cell granularity compared to control MSCs.

FIG. 2 provides graphs showing the effect of target and control MSCs on the proliferation of immune-activated T cells. Target and control MSCs were seeded in a 96-well plate at a concentration of 20 thousand cells/well. To control the number of reacting MSCs, their proliferation was halted by treatment with Mitomycin C. After removing the Mitomycin C, 200 thousand peripheral blood myonuclear cells (PBMCs), pre-stained with carboxyfluorescein succinimidyl ester (CFSE), were added into each well containing MSCs, as well as an additional control well without MSCs. The T cells in the PBMCs are then activated by adding T Cell TransAct beads (Miltenyi Biotec) into the wells conjugated with anti-CD3 anti-CD28 antibodies. Following 76 hours of co-culture, the suspension cells, consisting mainly of PBMCs, are removed from the wells, co-stained with anti-CD4 and anti-CD8 antibodies, and analyzed by flow cytometry for CFSE labeling against appropriate controls. The percent of activated CD8+ or CD4+ T cells is then calculated for each well as the ratio between the number of proliferative T cells—i.e., cells found in generations other than zero—and the total number of T cells analyzed (generation zero included).

FIG. 3 is a graph showing the effect of different concentrations of target and control MSCs on the proliferation of immune-activated T cells. Target and control MSCs were seeded in a 96-well plate at different concentrations of up to 30 thousand cells/well. To control the number of reacting MSCs, their proliferation was halted by treatment with Mitomycin C. After removing the Mitomycin C, 200 thousand PBMCs, pre-stained with CFSE, were added into each well containing MSCs, as well as additional control wells without MSCs. The T cells in the PBMCs are then activated by adding T Cell TransAct beads (Miltenyi Biotec) into the wells conjugated with anti-CD3 anti-CD28 antibodies. Following four days of co-culture, the suspension cells, consisting mainly of PBMCs, are removed from the wells, co-stained with anti-CD4, and analyzed by flow cytometry for CFSE labeling against appropriate controls. The percent of activated CD4+ T cells is then calculated for each well and plotted against the log 10 of the number of MSCs seeded in thousands (for example, x=1 denotes 10,000 cells/well). To calculate the IC50 values, a four-parameter logistic (4PL) regression curve was then fitted onto the data with high correlation (R2=0.9472 for control MSCs and R2=0.9953 for target MSCs).

FIG. 4A-4B are graphs demonstrating the effect of target and control MSCs on lungs' weights in an animal model for acute lung injury (ALI). Target MSCs were injected intravenously 6 hours after ALI induction in mice. Animals were sacrificed 18 hours post-treatment, and their lungs were harvested, weighed, and compared to lungs harvested from healthy untreated animals as well as model animals injected with Vehicle Control or control MSCs. Results are presented as (4A) individual lungs' weights (out-layer is encircled) and (4B) average lungs' weights±SEM (standard error of the mean) after removing the out-layers.

FIG. 5 provides bar graphs showing the immune cell counts in the BALF of ALI model animals post-treatment with target MSCs. Target MSCs were injected 6 hours post-ALI model induction, and animals were sacrificed 18 hours post-treatment. The animals' BALF cells were harvested, concentrated, pooled for every two animals, and subjected to CBC compared to BALF cells harvested from healthy untreated animals and model animals injected with the vehicle control item. Results are presented as the average counts±SEM of the total white blood cells (WBCs), lymphocytes, neutrophils, and monocytes.

FIG. 6 provides micrographs showing that soluble factors derived from target MSCs encourage the migration of endothelial and epithelial cells and inhibit the migration of fibroblasts in tissue cultures. Scale bar 200 μm.

FIGS. 7A-7C include bar graphs showing the therapeutic effect of the target MSCs as disclosed herein, in the context of coronavirus disease 2019 (COVID-19). (7A) Mortality rates among test and control patients at Visit 8 (one month after the first target MSCs dose or the equivalent time points for the control). (7B) Test and control patients' risk of deteriorating to mechanical ventilation. (7C) Average hospital length of stay (LoS) of patients having LoS >7 days. Two-sided p values were calculated using the Fisher Exact test (7A and 7B) or t-test (7C).

FIGS. 8A-8F include graphs showing that the target MSCs disclosed herein improve inflammation and tissue damage markers. (8A) CRP and (8B) CK levels were measured at Visit 6, the earliest of two weeks after the first target MSCs dose (Visit 2) or upon hospital release, or the equivalent time points for the control. The test and control groups started from similar median CRP and CK levels. (8C) Changes in control and test patients' LDH levels from Visit 1 (screening) to Visit 6. (8D) Area of test patients' diffuse pneumonia during Visits 1, 6, and 8 (one month after Visit 2). (8E) Blood oxygen saturation was measured during test patients' visits 1, 2-4 (upon or before receiving the first to third target MSCs dose), Visit 5 (the earliest of one week after Visit 2 or upon hospital release), and Visit 6. (8F) Test patients' blood lymphocytes levels (absolute) across Visits 1 and 6. Charts are presented as box-and-whiskers (according to the Tukey method). The p values were calculated using the Mann-Whitney test (8A, 8B, and 8C), Dunn's multiple comparisons (8D and 8E), or the Wilcoxon test (8F).

FIGS. 9A-9C include graphs showing that the target MSCs disclosed herein can reduce cytokine release syndrome (CRS) following cancer immunotherapy with CAR-T while well-tolerated and not obstructing the CAR-T anti-cancer activity. (9A) The levels of serum proinflammatory cytokines were measured in tumor-bearing NSG mice after CRS induction by injection of human PBMCs/CAR-Ts (or saline control) and treatment with target MSCs (or saline control). Experimental groups' designation: Control—not injected with PBMCs/CAR-Ts and not treated by target MSCs; CAR-T−CRS model animals, injected with PBMCs/CAR-Ts but not treated with target MSCs; MSCs—treated with target MSCs but not injected with PBMCs/CAR-Ts; and CAR-T+MSCs−CRS model animals treated with target MSCs. (9B) Relative change in body weight from the day of tumor induction (Day 0) and (9C) IVIS analysis of tumor burden (dorsal aspect) in the above four experimental groups. Statistical significance indicators: ns—not significant, *p<0.05, ***p<0.001, ****p<0.0001. Statistical tests: Holm-Šídák's multiple comparisons test (9A) and two-sided t-test (9C).

### DETAILED DESCRIPTION OF THE INVENTION

### Compositions

The present invention, in some embodiments, provides a composition comprising a cell population characterized by a gene expression profile. In some embodiments, the cell population in a composition as described herein is for transplantation, implantation, administration, and/or injection in a patient in need thereof. In some embodiments, the cell population is derived from cells grown in vitro.

In some embodiments, at least 80%, 90%, 95%, 99% or any range/value therebetween of the cell population/composition are MSCs. In some embodiments, the MSCs are derived from adipose stromal cells (ASC), a type of MSCs, primed/modified by a combination of biological and physical conditions to improve their potency, stability, and safety. In some embodiments, compositions comprising the cell population are useful for treating pulmonary manifestations of Covid-19. In some embodiments, the cell population/composition is composed of MSCs. In some embodiments, the term MSCs includes ASCs and vice versa.

In some embodiments, the mesenchymal cells disclosed herein are derived from: adipose tissue, umbilical cord, chorionic placenta, bone marrow, amniotic placenta, dental pulp, amniotic fluid, peripheral blood, synovium, synovial fluid, endometrium, skin, muscle, embryonic stem cells, induced pluripotent stem cells, or any combination thereof.

In some embodiments, “control MSC” and “control mesenchymal cells” are freshly dissected from a subject. In some embodiments, “control MSC” and “control mesenchymal cells” comprise naïve MSCs. In some embodiments, “control MSC” and “control mesenchymal cells” are not modified MSCs. In some embodiments, “control MSC” and “control mesenchymal cells” are unmodified MSCs. In some embodiments, “control MSC” and “control mesenchymal cells” comprise wildtype MSCs. In some embodiments, “control MSC” and “control mesenchymal cells” are not the target MSCs disclosed herein.

In some embodiments, the target mesenchymal cells disclosed herein are targeted or suitable for use in inhibition of an immune response in a subject in need thereof.

In some embodiments, the target mesenchymal cells of the invention are or comprise modified mesenchymal cells.

In some embodiments, the target MSCs population or composition alleviated edema in an acute lung injury (ALI) model by at least 60% and reduced the leukocytes' counts in the lung fluids by at least 40%.

In some embodiments, the composition comprising cells as described herein is administered in 2-4 or 1 IV administration to rescue subjects from a lethal ALI. In some embodiments, the composition comprising target MSCs as described herein inhibited the proliferation of activated T cells more profoundly than control MSCs. In some embodiments, the composition comprising target MSCs as described herein retained immunomodulatory capacity longer than unprimed/control MSCs representing a more stable product for transplantation/treatment with a longer shelf-life. In some embodiments, the composition comprising target MSCs as described herein possess improved hemocompatibility as evidenced by 50% lower levels of coagulation factor III at the mRNA, protein, and activity levels, as well as a >2-fold higher level of tissue factor pathway inhibitor compared to control MSCs, and unchanged levels of THBD encoding for thrombomodulin.

In some embodiments, the target MSCs are not subjected to genetic manipulation and/or genetic editing. In some embodiments, the targeted cells are obtained or achieved by modification comprising subjecting the cells to a combination of conditions selected from a group of conditions: hypoxia, nutrient depletion, serum depletion, oxidative stress, heat shock, over confluency, and exposing the cells to one or more biological agents such as growth factors, cytokines, and chemokines.

In some embodiments, the distinct features of the target cells include genes that are differently expressed between the target and the parallel or equivalent control cells as determined by RNA sequencing as well as morphological and functional changes as described below. In some embodiments of this invention, the target cells' distinct features include the upregulation of the two genes BCL2 and FGF7, despite the downregulation of the three genes HSP90, MTOR, and AKT1. In some embodiments of this invention, the target cells' distinct features include the downregulation of the gene PAK1 despite no downregulation of the gene THBD. In some embodiments of this invention, the target cells' distinct features include upregulation in the expression of the genes EDIL3 and AMOT. In some embodiments of this invention, the target cells' distinct features include the de novo expression or the upregulation of the gene IL6R.

In some embodiments of this invention, the target cells' distinct features include improved immunomodulatory capacity despite higher cell granularity and a higher proportion of cells at the G1 phase of the cell cycle.

In some embodiments, compositions comprising the target cells as described herein are useful for cell therapy or regenerative medicine for indications in the fields of traumatology, pneumology, neurology, cardiology, immunology, hepatology, endocrinology, dermatology, gastroenterology, nephrology, hematology, oncology, gynecology, psychiatry, urology, ophthalmology, odontology, and/or orthopedics.

In some embodiments, the novel target cells are characterized by a divergent gene expression profile in which the downstream genes BCL2 and FGF7 are upregulated despite the downregulation of commonly associated upstream genes HSP90, MTOR, and AKT1.

In some embodiments, the novel target cells uniquely exhibit activation patterns of pro-survival and potency genes seemingly independent of common regulators. Such a unique activation pattern is unexpectedly associated with the enhanced potency and stability of the target cells. In some embodiments, this enhancement might be attributed to the cells benefiting from both the divergent upregulation of genes such as BCL2 and FGF7 as well as the expected upregulation of other pro-survival genes such as SOD2, which are upregulated, expectedly, following the downregulation of AKT1 and HSP90.

In some embodiments, the downregulation of PAK1 in the present target cells is surprisingly not associated with the downregulation of THBD. In some embodiments, the present target cells express lower levels of coagulation factor 3 and higher levels of tissue factor pathway inhibitor, and uniquely unaffected levels of thrombomodulin, rendering the present target cells highly hemocompatible.

In some embodiments, the present target cells were shown in vitro and in vivo to exert more potent immunomodulatory and anti-inflammatory effects than unprimed/control MSCs. In some embodiments, the present target cells' enhanced capabilities are associated with the fact that the current cell population is enriched with cells having more than 8 and 37-fold upregulation of the genes EDIL3 and AMOT, respectively. In some embodiments, the present target cells were shown to dramatically overexpress AMOT and EDIL3 without genetic editing/modification of DNA.

In some embodiments, the invention provides a method for determining whether a composition is suitable for transplantation or administration in a patient in need thereof. In additional embodiments, the invention provides a panel of genes useful for determining whether a composition is suitable for transplantation or administration in a patient in need thereof.

The present invention is based, in part, on the finding that the cell population of the invention may be characterized by a gene expression signature of a plurality of genes. In some embodiments, the composition comprises target mesenchymal cells. In some embodiments, target mesenchymal cells comprise target mesenchymal stromal cells (MSC), mesenchymal stem cells, target fibroblast progenitor cells, differentiated/matured cells derived from mesenchymal cells, or any combination thereof.

In some embodiments, the composition comprises target mesenchymal cells characterized by overexpression of BCL2 and FGF7 genes compared to expression of the BCL2 and the FGF7 in control mesenchymal cells; and under-expression of HSP90AA1, MTOR, and AKT1 genes compared to expression of the HSP90AA1, the MTOR, and AKT1 genes in the control mesenchymal cells, wherein target comprises cells subjected to a combination of at least 2, 3, 4, 5, 6, 7, or 8 conditions selected from a group of conditions consisting of: (a) hypoxia, (b) serum-deprived, (c) oxidative stress, (d) hypothermia, (e) hyperthermia, (f) over confluency, (g) hydrostatic pressure, (h) dynamic or cyclic pressure, (i) shear forces, (j) agitation, (k) exposure to charged surfaces, and (1) under confluency.

In some embodiments, target mesenchymal cells are further characterized by under-expression of the PAK1 gene by at least 1.2-fold compared to expression of the PAK1 gene in control mesenchymal cells and less than 46% under-expression of THBD gene compared to expression of the THBD gene in control mesenchymal cells.

In some embodiments, target mesenchymal cells are further characterized by the under-expression of the PAK1 gene by at least 1.3-fold compared to control mesenchymal cells. In some embodiments, target mesenchymal cells are further characterized by the under-expression of the PAK1 gene by at least 1.4-fold compared to control mesenchymal cells. In some embodiments, target mesenchymal cells are further characterized by under-expression of the PAK1 gene by at least 1.5-fold compared to control mesenchymal cells. In some embodiments, target mesenchymal cells are further characterized by under-expression of the PAK1 gene by at least 1.6-fold compared to control mesenchymal cells. In some embodiments, target mesenchymal cells are further characterized by the under-expression of the PAK1 gene by at least 1.7-fold compared to control mesenchymal cells. In some embodiments, target mesenchymal cells are further characterized by the under-expression of the PAK1 gene by at least 1.8-fold compared to control mesenchymal cells. In some embodiments, target mesenchymal cells are further characterized by the under-expression of the PAK1 gene by at least 2-fold compared to control mesenchymal cells. In some embodiments, target mesenchymal cells are further characterized by the under-expression of the PAK1 gene by at least 2.2-fold compared to control mesenchymal cells. In some embodiments, target mesenchymal cells are further characterized by the under-expression of the PAK1 gene by at least 2.5-fold compared to control mesenchymal cells. In some embodiments, target mesenchymal cells are further characterized by the under-expression of the PAK1 gene by at least 2.8-fold compared to control mesenchymal cells. In some embodiments, target mesenchymal cells are further characterized by the under-expression of the PAK1 gene by at least 3-fold compared to control mesenchymal cells. In some embodiments, target mesenchymal cells are further characterized by the under-expression of the PAK1 gene by at least 3.5-fold compared to control mesenchymal cells.

In some embodiments, target mesenchymal cells are further characterized by less than 30% under-expression of the THBD gene compared to expression of the THBD gene in control mesenchymal cells. In some embodiments, target mesenchymal cells are further characterized by at least 34% under-expression of the THBD gene compared to expression of the THBD gene in control mesenchymal cells. In some embodiments, target mesenchymal cells are further characterized by at least 37% under-expression of the THBD gene compared to expression of the THBD gene in control mesenchymal cells. In some embodiments, target mesenchymal cells are further characterized by at least 40% under-expression of the THBD gene compared to expression of the THBD gene in control mesenchymal cells. In some embodiments, target mesenchymal cells are further characterized by at least 42% under-expression of the THBD gene compared to expression of the THBD gene in control mesenchymal cells. In some embodiments, target mesenchymal cells are further characterized by at least 45% under-expression of the THBD gene compared to expression of the THBD gene in control mesenchymal cells. In some embodiments, target mesenchymal cells are further characterized by at least 46% under-expression of the THBD gene compared to expression of the THBD gene in control mesenchymal cells. In some embodiments, target mesenchymal cells are further characterized by at least 48% under-expression of the THBD gene compared to expression of the THBD gene in control mesenchymal cells. In some embodiments, target mesenchymal cells are further characterized by at least 50% under-expression of the THBD gene compared to expression of the THBD gene in control mesenchymal cells. In some embodiments, target mesenchymal cells are further characterized by at least 55% under-expression of the THBD gene compared to expression of the THBD gene in control mesenchymal cells. In some embodiments, target mesenchymal cells are further characterized by at least 60% under-expression of the THBD gene compared to expression of the THBD gene in control mesenchymal cells. In some embodiments, target mesenchymal cells are further characterized by at least 65% under-expression of the THBD gene compared to expression of the THBD gene in control mesenchymal cells. In some embodiments, target mesenchymal cells are further characterized by at least 70% under-expression of the THBD gene compared to expression of the THBD gene in control mesenchymal cells.

In some embodiments, the target mesenchymal cells are further characterized by overexpression of the EDIL3 gene, AMOT gene, or their combination, compared to expression of the EDIL3, AMOT, or both in control mesenchymal cells.

In some embodiments, the target mesenchymal cells are further characterized by 4-fold overexpression of the EDIL3 gene compared to the expression of EDIL3 in control mesenchymal cells. In some embodiments, the target mesenchymal cells are further characterized by 4.2-fold overexpression of the EDIL3 gene compared to the expression of EDIL3 in control mesenchymal cells. In some embodiments, the target mesenchymal cells are further characterized by 4.5-fold overexpression of the EDIL3 gene compared to the expression of EDIL3 in control mesenchymal cells. In some embodiments, the target mesenchymal cells are further characterized by 4.7-fold overexpression of the EDIL3 gene compared to the expression of EDIL3 in control mesenchymal cells. In some embodiments, the target mesenchymal cells are further characterized by 5-fold overexpression of the EDIL3 gene compared to the expression of EDIL3 in control mesenchymal cells. In some embodiments, the target mesenchymal cells are further characterized by 5.2-fold overexpression of the EDIL3 gene compared to the expression of EDIL3 in control mesenchymal cells. In some embodiments, the target mesenchymal cells are further characterized by 5.5-fold overexpression of the EDIL3 gene compared to the expression of EDIL3 in control mesenchymal cells. In some embodiments, the target mesenchymal cells are further characterized by 5.7-fold overexpression of the EDIL3 gene compared to the expression of EDIL3 in control mesenchymal cells. In some embodiments, the target mesenchymal cells are further characterized by 6-fold overexpression of the EDIL3 gene compared to the expression of EDIL3 in control mesenchymal cells. In some embodiments, the target mesenchymal cells are further characterized by 6.4-fold overexpression of the EDIL3 gene compared to the expression of EDIL3 in control mesenchymal cells. In some embodiments, the target mesenchymal cells are further characterized by 6.8-fold overexpression of the EDIL3 gene compared to the expression of EDIL3 in control mesenchymal cells. In some embodiments, the target mesenchymal cells are further characterized by 7-fold overexpression of the EDIL3 gene compared to the expression of EDIL3 in control mesenchymal cells. In some embodiments, the target mesenchymal cells are further characterized by 7.2-fold overexpression of the EDIL3 gene compared to the expression of EDIL3 in control mesenchymal cells. In some embodiments, the target mesenchymal cells are further characterized by 7.4-fold overexpression of the EDIL3 gene compared to the expression of EDIL3 in control mesenchymal cells. In some embodiments, the target mesenchymal cells are further characterized by 7.6-fold overexpression of the EDIL3 gene compared to the expression of EDIL3 in control mesenchymal cells. In some embodiments, the target mesenchymal cells are further characterized by 7.8-fold overexpression of the EDIL3 gene compared to the expression of EDIL3 in control mesenchymal cells. In some embodiments, the target mesenchymal cells are further characterized by 8-fold overexpression of the EDIL3 gene compared to the expression of EDIL3 in control mesenchymal cells. In some embodiments, the target mesenchymal cells are further characterized by 8.2-fold overexpression of the EDIL3 gene compared to the expression of EDIL3 in control mesenchymal cells. In some embodiments, the target mesenchymal cells are further characterized by 8.5-fold overexpression of the EDIL3 gene compared to the expression of EDIL3 in control mesenchymal cells. In some embodiments, the target mesenchymal cells are further characterized by 8.7-fold overexpression of the EDIL3 gene compared to the expression of EDIL3 in control mesenchymal cells. In some embodiments, the target mesenchymal cells are further characterized by 9-fold overexpression of the EDIL3 gene compared to the expression of EDIL3 in control mesenchymal cells. In some embodiments, the target mesenchymal cells are further characterized by 9.5-fold overexpression of the EDIL3 gene compared to the expression of EDIL3 in control mesenchymal cells. In some embodiments, the target mesenchymal cells are further characterized by 10-fold overexpression of the EDIL3 gene compared to the expression of EDIL3 in control mesenchymal cells.

In some embodiments, the target mesenchymal cells are further characterized by at least 4-fold overexpression of the AMOT gene compared to expression of AMOT in control mesenchymal cells. In some embodiments, the target mesenchymal cells are further characterized by at least 4.2-fold overexpression of the AMOT gene compared to the expression of AMOT in control mesenchymal cells. In some embodiments, the target mesenchymal cells are further characterized by at least 4.5-fold overexpression of the AMOT gene compared to the expression of AMOT in control mesenchymal cells. In some embodiments, the target mesenchymal cells are further characterized by 4.7-fold overexpression of the AMOT gene compared to the expression of AMOT in control mesenchymal cells. In some embodiments, the target mesenchymal cells are further characterized by at least 5-fold overexpression of the AMOT gene compared to expression of AMOT in control mesenchymal cells. In some embodiments, the target mesenchymal cells are further characterized by at least 5.2-fold overexpression of the AMOT gene compared to the expression of AMOT in control mesenchymal cells. In some embodiments, the target mesenchymal cells are further characterized by 5.5-fold overexpression of the AMOT gene compared to the expression of AMOT in control mesenchymal cells. In some embodiments, the target mesenchymal cells are further characterized by at least 5.7-fold overexpression of the AMOT gene compared to the expression of AMOT in control mesenchymal cells. In some embodiments, the target mesenchymal cells are further characterized by at least 6-fold overexpression of the AMOT gene compared to the expression of AMOT in control mesenchymal cells. In some embodiments, the target mesenchymal cells are further characterized by at least 6.4-fold overexpression of the AMOT gene compared to the expression of AMOT in control mesenchymal cells. In some embodiments, the target mesenchymal cells are further characterized by at least 6.8-fold overexpression of the AMOT gene compared to the expression of AMOT in control mesenchymal cells. In some embodiments, the target mesenchymal cells are further characterized by at least 7-fold overexpression of the AMOT gene compared to the expression of AMOT in control mesenchymal cells. In some embodiments, the target mesenchymal cells are further characterized by at least 7.2-fold overexpression of the AMOT gene compared to the expression of AMOT in control mesenchymal cells. In some embodiments, the target mesenchymal cells are further characterized by at least 7.4-fold overexpression of the AMOT gene compared to the expression of AMOT in control mesenchymal cells. In some embodiments, the target mesenchymal cells are further characterized by at least 7.6-fold overexpression of the AMOT gene compared to the expression of AMOT in control mesenchymal cells. In some embodiments, the target mesenchymal cells are further characterized by at least 7.8-fold overexpression of the AMOT gene compared to the expression of AMOT in control mesenchymal cells. In some embodiments, the target mesenchymal cells are further characterized by 8-fold overexpression of the AMOT gene compared to the expression of AMOT in control mesenchymal cells. In some embodiments, the target mesenchymal cells are further characterized by at least 8.2-fold overexpression of the AMOT gene compared to the expression of AMOT in control mesenchymal cells. In some embodiments, the target mesenchymal cells are further characterized by 8.5-fold overexpression of the AMOT gene compared to the expression of AMOT in control mesenchymal cells. In some embodiments, the target mesenchymal cells are further characterized by 8.7-fold overexpression of the AMOT gene compared to the expression of AMOT in control mesenchymal cells. In some embodiments, the target mesenchymal cells are further characterized by at least 9-fold overexpression of the AMOT gene compared to the expression of AMOT in control mesenchymal cells. In some embodiments, the target mesenchymal cells are further characterized by at least 9.5-fold overexpression of the AMOT gene compared to the expression of AMOT in control mesenchymal cells. In some embodiments, the target mesenchymal cells are further characterized by at least 10-fold overexpression of the AMOT gene compared to the expression of AMOT in control mesenchymal cells. In some embodiments, the target mesenchymal cells are further characterized by at least 50, 100, 150, 200, 250, 300, or any value therebetween, fold overexpression of AMOT gene compared to expression of AMOT in control mesenchymal cells.

In some embodiments, target mesenchymal cells are further characterized by over-expression of the BCL2 gene by at least 1.1-fold compared to control mesenchymal cells. In some embodiments, target mesenchymal cells are further characterized by over-expression of the BCL2 gene by at least 1.2-fold compared to control mesenchymal cells. In some embodiments, target mesenchymal cells are further characterized by over-expression of the BCL2 gene by at least 1.25-fold compared to control mesenchymal cells. In some embodiments, target mesenchymal cells are further characterized by over-expression of the BCL2 gene by at least 1.3-fold compared to control mesenchymal cells. In some embodiments, target mesenchymal cells are further characterized by over-expression of the BCL2 gene by at least 1.4-fold compared to control mesenchymal cells. In some embodiments, target mesenchymal cells are further characterized by over-expression of the BCL2 gene by at least 1.5-fold compared to control mesenchymal cells. In some embodiments, target mesenchymal cells are further characterized by over-expression of the BCL2 gene by at least 1.6-fold compared to control mesenchymal cells. In some embodiments, target mesenchymal cells are further characterized by over-expression of the BCL2 gene by at least 1.7-fold compared to control mesenchymal cells. In some embodiments, target mesenchymal cells are further characterized by over-expression of the BCL2 gene by at least 1.8-fold compared to control mesenchymal cells. In some embodiments, target mesenchymal cells are further characterized by over-expression of the BCL2 gene by at least 2-fold compared to control mesenchymal cells. In some embodiments, target mesenchymal cells are further characterized by over-expression of the BCL2 gene by at least 2.2-fold compared to control mesenchymal cells. In some embodiments, target mesenchymal cells are further characterized by over-expression of the BCL2 gene by at least 2.5-fold compared to control mesenchymal cells. In some embodiments, target mesenchymal cells are further characterized by over-expression of the BCL2 gene by at least 2.8-fold compared to control mesenchymal cells. In some embodiments, target mesenchymal cells are further characterized by over-expression of the BCL2 gene by at least 3-fold compared to control mesenchymal cells. In some embodiments, target mesenchymal cells are further characterized by over-expression of the BCL2 gene by at least 3.5-fold compared to control mesenchymal cells. In some embodiments, target mesenchymal cells are further characterized by over-expression of the BCL2 gene by at least 3.6-fold compared to control mesenchymal cells. In some embodiments, target mesenchymal cells are further characterized by over-expression of the BCL2 gene by at least 3.7-fold compared to control mesenchymal cells. In some embodiments, target mesenchymal cells are further characterized by over-expression of the BCL2 gene by at least 3.8-fold compared to control mesenchymal cells. In some embodiments, target mesenchymal cells are further characterized by over-expression of the BCL2 gene by at least 4-fold compared to control mesenchymal cells. In some embodiments, target mesenchymal cells are further characterized by over-expression of the BCL2 gene by at least 4.5-fold compared to control mesenchymal cells. In some embodiments, target mesenchymal cells are further characterized by over-expression of the BCL2 gene by at least 5-fold compared to control mesenchymal cells.

In some embodiments, the target mesenchymal cells are further characterized by 4-fold overexpression of the FGF7 gene compared to expression of FGF7 in control mesenchymal cells. In some embodiments, the target mesenchymal cells are further characterized by 4.2-fold overexpression of the FGF7 gene compared to the expression of FGF7 in control mesenchymal cells. In some embodiments, the target mesenchymal cells are further characterized by 4.5-fold overexpression of the FGF7 gene compared to the expression of FGF7 in control mesenchymal cells. In some embodiments, the target mesenchymal cells are further characterized by 4.7-fold overexpression of the FGF7 gene compared to the expression of FGF7 in control mesenchymal cells. In some embodiments, the target mesenchymal cells are further characterized by 5-fold overexpression of the FGF7 gene compared to the expression of FGF7 in control mesenchymal cells. In some embodiments, the target mesenchymal cells are further characterized by 5.2-fold overexpression of the FGF7 gene compared to the expression of FGF7 in control mesenchymal cells. In some embodiments, the target mesenchymal cells are further characterized by 5.5-fold overexpression of the FGF7 gene compared to the expression of FGF7 in control mesenchymal cells. In some embodiments, the target mesenchymal cells are further characterized by 5.7-fold overexpression of the FGF7 gene compared to the expression of FGF7 in control mesenchymal cells. In some embodiments, the target mesenchymal cells are further characterized by 6-fold overexpression of the FGF7 gene compared to expression of FGF7 in control mesenchymal cells. In some embodiments, the target mesenchymal cells are further characterized by 6.4-fold overexpression of the FGF7 gene compared to the expression of FGF7 in control mesenchymal cells. In some embodiments, the target mesenchymal cells are further characterized by 6.8-fold overexpression of the FGF7 gene compared to the expression of FGF7 in control mesenchymal cells.

In some embodiments, the target mesenchymal cells are further characterized by 7-fold overexpression of the FGF7 gene compared to expression of FGF7 in control mesenchymal cells. In some embodiments, the target mesenchymal cells are further characterized by 7.2-fold overexpression of the FGF7 gene compared to the expression of FGF7 in control mesenchymal cells. In some embodiments, the target mesenchymal cells are further characterized by 7.4-fold overexpression of the FGF7 gene compared to the expression of FGF7 in control mesenchymal cells. In some embodiments, the target mesenchymal cells are further characterized by 7.6-fold overexpression of the FGF7 gene compared to the expression of FGF7 in control mesenchymal cells. In some embodiments, the target mesenchymal cells are further characterized by 7.8-fold overexpression of the FGF7 gene compared to the expression of FGF7 in control mesenchymal cells. In some embodiments, the target mesenchymal cells are further characterized by 8-fold overexpression of the FGF7 gene compared to expression of FGF7 in control mesenchymal cells. In some embodiments, the target mesenchymal cells are further characterized by 9-fold overexpression of the FGF7 gene compared to expression of FGF7 in control mesenchymal cells. In some embodiments, the target mesenchymal cells are further characterized by 9.5-fold overexpression of the FGF7 gene compared to the expression of FGF7 in control mesenchymal cells. In some embodiments, the target mesenchymal cells are further characterized by 10-fold overexpression of the FGF7 gene compared to the expression of FGF7 in control mesenchymal cells.

In some embodiments, target mesenchymal cells are further characterized by the under-expression of the HSP90AA1 gene by at least 1.2-fold compared to control mesenchymal cells. In some embodiments, target mesenchymal cells are further characterized by the under-expression of the HSP90AA1 gene by at least 1.3-fold compared to control mesenchymal cells. In some embodiments, target mesenchymal cells are further characterized by the under-expression of the HSP90AA1 gene by at least 1.4-fold compared to control mesenchymal cells. In some embodiments, target mesenchymal cells are further characterized by the under-expression of the HSP90AA1 gene by at least 1.5-fold compared to control mesenchymal cells. In some embodiments, target mesenchymal cells are further characterized by the under-expression of the HSP90AA1 gene by at least 1.6-fold compared to control mesenchymal cells. In some embodiments, target mesenchymal cells are further characterized by the under-expression of the HSP90AA1 gene by at least 1.7-fold compared to control mesenchymal cells. In some embodiments, target mesenchymal cells are further characterized by the under-expression of the HSP90AA1 gene by at least 1.8-fold compared to control mesenchymal cells. In some embodiments, target mesenchymal cells are further characterized by the under-expression of the HSP90AA1 gene by at least 2.2-fold compared to control mesenchymal cells. In some embodiments, target mesenchymal cells are further characterized by the under-expression of the HSP90AA1 gene by at least 2.4-fold compared to control mesenchymal cells. In some embodiments, target mesenchymal cells are further characterized by the under-expression of the HSP90AA1 gene by at least 2.5-fold compared to control mesenchymal cells. In some embodiments, target mesenchymal cells are further characterized by the under-expression of the HSP90AA1 gene by at least 2.6-fold compared to control mesenchymal cells. In some embodiments, target mesenchymal cells are further characterized by the under-expression of the HSP90AA1 gene by at least 2.7-fold compared to control mesenchymal cells. In some embodiments, target mesenchymal cells are further characterized by the under-expression of the HSP90AA1 gene by at least 2.8-fold compared to control mesenchymal cells. In some embodiments, target mesenchymal cells are further characterized by the under-expression of the HSP90AA1 gene by at least 3-fold compared to control mesenchymal cells. In some embodiments, target mesenchymal cells are further characterized by the under-expression of the HSP90AA1 gene by at least 3.2-fold compared to control mesenchymal cells. In some embodiments, target mesenchymal cells are further characterized by the under-expression of the HSP90AA1 gene by at least 3.5-fold compared to control mesenchymal cells. In some embodiments, target mesenchymal cells are further characterized by the under-expression of the HSP90AA1 gene by at least 3.8-fold compared to control mesenchymal cells. In some embodiments, target mesenchymal cells are further characterized by the under-expression of the HSP90AA1 gene by at least 4-fold compared to control mesenchymal cells. In some embodiments, target mesenchymal cells are further characterized by under-expression of the HSP90AA1 gene by at least 4.2, 4.4, 4.6, 5, 5.4, 6, 6.5, 7, 8 or any range/value therebetween, fold compared to control mesenchymal cells.

In some embodiments, target mesenchymal cells are further characterized by under-expression of the HSP90AA1 gene of no more than 3.5-fold compared to control mesenchymal cells. In some embodiments, target mesenchymal cells are further characterized by under-expression of the HSP90AA1 gene of no more than 4-fold compared to control mesenchymal cells. In some embodiments, target mesenchymal cells are further characterized by under-expression of the HSP90AA1 gene of no more than 5-fold compared to control mesenchymal cells. In some embodiments, target mesenchymal cells are further characterized by under-expression of the HSP90AA1 gene of no more than 6-fold compared to control mesenchymal cells. In some embodiments, target mesenchymal cells are further characterized by under-expression of the HSP90AA1 gene of no more than 8-fold compared to control mesenchymal cells.

In some embodiments, target mesenchymal cells are further characterized by the under-expression of the MTOR gene by at least 1.2-fold compared to control mesenchymal cells. In some embodiments, target mesenchymal cells are further characterized by the under-expression of the MTOR gene by at least 1.3-fold compared to control mesenchymal cells. In some embodiments, target mesenchymal cells are further characterized by the under-expression of the MTOR gene by at least 1.4-fold compared to control mesenchymal cells. In some embodiments, target mesenchymal cells are further characterized by the under-expression of the MTOR gene by at least 1.5-fold compared to control mesenchymal cells. In some embodiments, target mesenchymal cells are further characterized by the under-expression of the MTOR gene by at least 1.6-fold compared to control mesenchymal cells. In some embodiments, target mesenchymal cells are further characterized by the under-expression of the MTOR gene by at least 1.7-fold compared to control mesenchymal cells. In some embodiments, target mesenchymal cells are further characterized by the under-expression of the MTOR gene by at least 1.8-fold compared to control mesenchymal cells. In some embodiments, target mesenchymal cells are further characterized by the under-expression of the MTOR gene by at least 2.2-fold compared to control mesenchymal cells. In some embodiments, target mesenchymal cells are further characterized by the under-expression of the MTOR gene by at least 2.4-fold compared to control mesenchymal cells. In some embodiments, target mesenchymal cells are further characterized by the under-expression of the MTOR gene by at least 2.5-fold compared to control mesenchymal cells. In some embodiments, target mesenchymal cells are further characterized by the under-expression of the MTOR gene by at least 2.6-fold compared to control mesenchymal cells. In some embodiments, target mesenchymal cells are further characterized by the under-expression of the MTOR gene by at least 2.7-fold compared to control mesenchymal cells. In some embodiments, target mesenchymal cells are further characterized by the under-expression of the MTOR gene by at least 2.8-fold compared to control mesenchymal cells. In some embodiments, target mesenchymal cells are further characterized by the under-expression of the MTOR gene by at least 3-fold compared to control mesenchymal cells. In some embodiments, target mesenchymal cells are further characterized by the under-expression of the MTOR gene by at least 3.2-fold compared to control mesenchymal cells. In some embodiments, target mesenchymal cells are further characterized by the under-expression of the MTOR gene by at least 3.5-fold compared to control mesenchymal cells. In some embodiments, target mesenchymal cells are further characterized by the under-expression of the MTOR gene by at least 3.8-fold compared to control mesenchymal cells. In some embodiments, target mesenchymal cells are further characterized by the under-expression of the MTOR gene by at least 4-fold compared to control mesenchymal cells.

In some embodiments, target mesenchymal cells are further characterized by under-expression of the MTOR gene of no more than 3.5-fold compared to control mesenchymal cells. In some embodiments, target mesenchymal cells are further characterized by under-expression of the MTOR gene of no more than 4-fold compared to control mesenchymal cells. In some embodiments, target mesenchymal cells are further characterized by under-expression of the MTOR gene of no more than 5-fold compared to control mesenchymal cells. In some embodiments, target mesenchymal cells are further characterized by under-expression of the MTOR gene of no more than 6-fold compared to control mesenchymal cells. In some embodiments, target mesenchymal cells are further characterized by under-expression of the MTOR gene of no more than 8-fold compared to control mesenchymal cells.

In some embodiments, target mesenchymal cells are further characterized by the under-expression of the AKT1 gene by at least 1.2-fold compared to control mesenchymal cells. In some embodiments, target mesenchymal cells are further characterized by the under-expression of the AKT1 gene by at least 1.3-fold compared to control mesenchymal cells. In some embodiments, target mesenchymal cells are further characterized by the under-expression of the AKT1 gene by at least 1.4-fold compared to control mesenchymal cells. In some embodiments, target mesenchymal cells are further characterized by the under-expression of the AKT1 gene by at least 1.5-fold compared to control mesenchymal cells. In some embodiments, target mesenchymal cells are further characterized by the under-expression of the AKT1 gene by at least 1.6-fold compared to control mesenchymal cells. In some embodiments, target mesenchymal cells are further characterized by the under-expression of the AKT1 gene by at least 1.7-fold compared to control mesenchymal cells. In some embodiments, target mesenchymal cells are further characterized by the under-expression of the AKT1 gene by at least 1.8-fold compared to control mesenchymal cells. In some embodiments, target mesenchymal cells are further characterized by the under-expression of the AKT1 gene by at least 2.2-fold compared to control mesenchymal cells. In some embodiments, target mesenchymal cells are further characterized by the under-expression of the AKT1 gene by at least 2.4-fold compared to control mesenchymal cells. In some embodiments, target mesenchymal cells are further characterized by the under-expression of the AKT1 gene by at least 2.5-fold compared to control mesenchymal cells. In some embodiments, target mesenchymal cells are further characterized by the under-expression of the AKT1 gene by at least 1.6-fold compared to control mesenchymal cells. In some embodiments, target mesenchymal cells are further characterized by the under-expression of the AKT1 gene by at least 2.7-fold compared to control mesenchymal cells. In some embodiments, target mesenchymal cells are further characterized by the under-expression of the AKT1 gene by at least 2.8-fold compared to control mesenchymal cells. In some embodiments, target mesenchymal cells are further characterized by the under-expression of the AKT1 gene by at least 3-fold compared to control mesenchymal cells. In some embodiments, target mesenchymal cells are further characterized by the under-expression of the AKT1 gene by at least 3.2-fold compared to control mesenchymal cells. In some embodiments, target mesenchymal cells are further characterized by the under-expression of the AKT1 gene by at least 3.5-fold compared to control mesenchymal cells. In some embodiments, target mesenchymal cells are further characterized by the under-expression of the AKT1 gene by at least 3.8-fold compared to control mesenchymal cells. In some embodiments, target mesenchymal cells are further characterized by the under-expression of the AKT1 gene by at least 4-fold compared to control mesenchymal cells.

In some embodiments, target mesenchymal cells are further characterized by under-expression of the AKT1 gene of no more than 3.5-fold compared to control mesenchymal cells. In some embodiments, target mesenchymal cells are further characterized by under-expression of the AKT1 gene of no more than 4-fold compared to control mesenchymal cells. In some embodiments, target mesenchymal cells are further characterized by under-expression of the AKT1 gene of no more than 5-fold compared to control mesenchymal cells. In some embodiments, target mesenchymal cells are further characterized by under-expression of the AKT1 gene of no more than 6-fold compared to control mesenchymal cells. In some embodiments, target mesenchymal cells are further characterized by under-expression of the AKT1 gene of no more than 8-fold compared to control mesenchymal cells.

In some embodiments, the target mesenchymal cells are further characterized by overexpression or de novo expression of the IL6R gene compared to the expression of IL6R in control mesenchymal cells.

In some embodiments, the target mesenchymal cells are further characterized by increased cell granularity compared to control mesenchymal cells. In some embodiments, the target mesenchymal cells are further characterized by a larger proportion of cells in the G1 phase of the cell cycle compared to control mesenchymal cells.

In some embodiments, at least 15% of the target mesenchymal cells within the composition are in the G1 phase of the cell cycle.

In some embodiments, at least 15% of the target mesenchymal cells within the composition are in the G1 phase of the cell cycle. In some embodiments, at least 20% of the target mesenchymal cells within the composition are in the G1 phase of the cell cycle. In some embodiments, at least 25% of the target mesenchymal cells within the composition are in the G1 phase of the cell cycle. In some embodiments, at least 30% of the target mesenchymal cells within the composition are in the G1 phase of the cell cycle. In some embodiments, at least 35% of the target mesenchymal cells within the composition are in the G1 phase of the cell cycle. In some embodiments, at least 40% of the target mesenchymal cells within the composition are in the G1 phase of the cell cycle. In some embodiments, at least 45% of the target mesenchymal cells within the composition are in the G1 phase of the cell cycle. In some embodiments, at least 50% of the target mesenchymal cells within the composition are in the G1 phase of the cell cycle. In some embodiments, at least 15% of the target mesenchymal cells within the composition are in the G1 phase of the cell cycle. In some embodiments, at least 55% of the target mesenchymal cells within the composition are in the G1 phase of the cell cycle. In some embodiments, at least 60% of the target mesenchymal cells within the composition are in the G1 phase of the cell cycle. In some embodiments, at least 65% of the target mesenchymal cells within the composition are in the G1 phase of the cell cycle. In some embodiments, at least 70% of the target mesenchymal cells within the composition are in the G1 phase of the cell cycle. In some embodiments, at least 75% of the target mesenchymal cells within the composition are in the G1 phase of the cell cycle.

### Methods

According to some embodiments, there is provided a method for inhibiting an immune response in a subject in need thereof, comprising administering to the subject a composition as described herein comprising cells described herein, thereby inhibiting an immune response in a subject in need thereof.

In some embodiments, there is provided a method for treating or preventing acute respiratory distress syndrome.

In some embodiments, there is provided a method for treating or preventing cytokine storm or cytokine release syndrome in a subject in need thereof. In some embodiments, the cytokine storm or cytokine release syndrome is induced by a pulmonary pathogen. In some embodiments, the cytokine storm or cytokine release syndrome is induced by a virus. In some embodiments, the cytokine storm or cytokine release syndrome is induced by another form of therapeutic intervention (e.g., immunotherapy). In some embodiments, the virus comprises a coronavirus. In some embodiments, the virus or coronavirus comprises SARS-COV-2.

In some embodiments, there is provided a method for treating or preventing pulmonary manifestations of Covid-19.

In some embodiments, the immune response is an adaptive immune response, an innate immune response, or both. In some embodiments, the subject is afflicted with a cytokine storm, cytokine release syndrome, or sepsis or at risk of developing a cytokine storm, cytokine release syndrome, sepsis, or SIRS. In some embodiments, inhibiting comprises the inhibiting of CD4+ cells, CD8+ cells, or both. In some embodiments, “inhibiting” comprises inhibiting cytokine production in the subject. In some embodiments, “inhibiting” comprises inhibiting cytokine secretion in the subject. In some embodiments, “inhibiting” comprises inhibiting cytokine production and secretion in the subject. In some embodiments, “inhibiting” comprises inhibiting the effect of cytokines in the subject. In some embodiments, the cytokine is a pro-inflammatory cytokine.

In some embodiments, inhibiting comprises inhibiting cytotoxic activity.

As used herein, the terms “cytokine release syndrome (CRS)” and “cytokine storm” refer to the dysregulated production and/or secretion of pro-inflammatory cytokines leading to disease. Often, a cytokine storm or cascade is referred to as being part of a sequence because one cytokine typically leads to the production of multiple other cytokines that can reinforce and amplify the immune response.

In some embodiments, the cytokine is selected from interferon-gamma (IFNγ), tumor necrosis factor-alpha (TNFα), interleukin (IL)-10, IL-6, IL-4, IL-2, or any combination thereof.

In some embodiments, the method is for inhibiting an innate and/or adaptive immune response in a subject. In some embodiments, the subject is afflicted with an autoimmune disease or with a disease manifested and/or caused by a component of an immune response.

In some embodiments, the method for inhibiting an innate immune response comprises inhibiting macrophages, neutrophils, dendritic cells, mast cells, Natural killer cells, basophils, eosinophils, or any combination thereof. In some embodiments, the method for inhibiting an innate immune response comprises inhibiting cytokines-dependent nonspecific immunity of leukocytes, HLA-independent pathogen-killing cells, phagocytosis, or any combination thereof. In some embodiments, the method for inhibiting an innate immune response comprises inhibiting inflammation.

In some embodiments, the method for inhibiting an innate immune response comprises inhibiting the production of cytokines, chemokines, and interleukins associated with innate immunity. In some embodiments, the method for inhibiting an innate immune response comprises inhibiting the production of an interferon, a TNF molecule, an interleukin (such as 12 and 18) 12 and 18, or any combination thereof. In some embodiments, the method for inhibiting an innate immune response comprises inhibiting the complement system.

In some embodiments, the method further comprises reducing or inhibiting inflammation.

In some embodiments, the method comprises reducing C-reactive protein (CRP) levels, creatine kinase (CK) levels, lactate dehydrogenase (LDH) levels, or any combination thereof in the subject. In some embodiments, levels comprise blood and/or serum levels. In some embodiments, the method further comprises a step of determining CRP levels, CK levels, LDH levels, or any combination thereof in the subject or in a biological sample, e.g., blood and/or serum, obtained or derived therefrom.

In some embodiments, the method comprises increasing blood oxygen saturation in the subject. In some embodiments, the method comprises reducing diffuse pneumonia in the subject. In one embodiment, diffuse pneumonia is determined as % of the lungs area. In some embodiments, the method comprises increasing blood lymphocyte levels in the subject.

In some embodiments, the method as described herein is for inhibiting adaptive immune response in a subject in need thereof, comprising administering the composition comprising cells as described herein.

In one embodiment, the subject is afflicted with “anti-infectious-virus antibodies,” which are antibodies that bind a viral antigen belonging to an infectious virus. In one embodiment, the subject is afflicted with “anti-self-antigen antibodies”. In one embodiment, the subject is afflicted with an autoimmune disease. In one embodiment, the subject is afflicted with graft versus host disease. In one embodiment, the subject is at risk of developing antibodies against self-antigen. In one embodiment, the subject is at risk of developing antibodies against a transplanted cell, tissue, and/or organ. In one embodiment, the subject is destined for organ or cell transplantation. In one embodiment, the subject is a patient having a transplanted cell or tissue. In one embodiment, the subject is a patient who has undergone transplantation before or after developing an immune response against the transplanted cells, tissue, and/or organ. In one embodiment, the subject is a patient undergoing immunotherapy.

In one embodiment, inhibiting an adaptive immune response comprises inhibiting antigen-presenting cells (APCs). In one embodiment, inhibiting antigen-presenting cells comprises inhibiting APCs' ability to present antigens. In one embodiment, inhibiting antigen-presenting cells comprises inhibiting APCs to present antigens of an infectious virus on their MHC II molecule. In one embodiment, inhibiting an adaptive immune response comprises inhibiting NK cells, T cells, B cells, or all. In one embodiment, inhibiting an adaptive immune response comprises inhibiting T-helper cells. In one embodiment, inhibiting an adaptive immune response comprises inhibiting the number of memory cells. In one embodiment, inhibiting an adaptive immune response comprises inhibiting the number of T-memory cells. In one embodiment, inhibiting an adaptive immune response comprises inhibiting the number of B-memory cells.

In one embodiment, inhibiting an adaptive immune response is inhibiting an adaptive immune response against an antigen present within the subject. In one embodiment, inhibiting an adaptive immune response is inhibiting an adaptive immune response against a viral antigen, wherein the virus comprising the viral antigen is present within the subject. In one embodiment, inhibiting an adaptive immune response is inhibiting the proliferation, maturation, or both of CD8+ cells. In one embodiment, inhibiting an adaptive immune response is inhibiting the proliferation, maturation, or both of CD4+ cells. In one embodiment, inhibiting an adaptive immune response is inhibiting the proliferation, maturation, or both of both CD4+ cells and CD8+ cells.

In one embodiment, inhibiting an adaptive immune response is inhibiting the production of neutralizing anti-self-antigen antibodies. In one embodiment, inhibiting an adaptive immune response is inhibiting cytokine or pro-inflammatory cytokine production in the subject.

In one embodiment, a method as described herein comprises initially selecting the subject in need of immune response inhibition.

The term “subject” as used herein refers to an animal, e.g., a non-human mammal or a human. Non-human animal subjects may also include prenatal forms of animals, such as embryos or fetuses. Non-limiting examples of non-human animals include horse, cow, camel, goat, sheep, dog, cat, monkey, non-human primate, mouse, rat, rabbit, hamster, guinea pig, and pig. In one embodiment, the subject is a human. Human subjects may also include fetuses.

In one embodiment, provided herein is a method for inducing tissue regeneration in an injured tissue or a degenerated tissue in a subject in need thereof, comprising administering to the subject the composition comprising the cells as described herein, thereby inducing tissue regeneration in an injured tissue or a degenerated tissue in a subject in need thereof. In one embodiment, tissue regeneration comprises induction of wound healing, reendothelization, controlling fibrosis, reepithelization, cell proliferation, cell migration, or any combination thereof. In one embodiment, tissue regeneration comprises inhibiting tissue fibrosis.

In one embodiment, provided herein is a method for inducing cell migration in endothelial cell, epithelial cell, or both in a subject in need thereof, comprising administering to the subject the composition comprising the cells as described herein. In one embodiment, provided herein is a method for inhibiting or controlling the migration of fibroblastic cells in a subject in need thereof, comprising administering to the subject the composition comprising the cells as described herein.

As used herein, the terms implanting or implantation, transplanting or transplantation, administering or administration, injecting or injection, delivering or delivery all refer to the process of providing the composition disclosed herein to the site of treatment and would be understood by a person of ordinary skill in the art to have the same meaning, depending on the composition properties and procedure employed for carrying out the delivery of tissue to the site. These terms can be used interchangeably and are in no way limiting to the method of the invention.

As used herein, the terms “gene expression profile”, “gene expression signature” or “gene expression fingerprint” are interchangeable and refer to the pattern of gene expression modulation/difference, including the increase or decrease of expression, exhibited by the target cell population of the invention compared to untarget populations of cells of the same origin/source. The profile or fingerprint includes the relative degree of increase or decrease of expression of the “differentially expressed genes” (DEG) compared to control.

The terms “differentially expressed genes”, “DEGs, “differential gene expression” and their synonyms, which are used interchangeably, refer to a gene whose expression is upregulated or downregulated to a higher or lower level in a selected population of target cells compared to a control (control). It is also understood that a differentially expressed gene may be either activated or inhibited at the nucleic acid level or protein level or may be subject to alternative splicing to result in a different polypeptide product. Such differences, for example, may be evidenced by a change in mRNA levels, surface expression, secretion, or another partitioning of a polypeptide.

As used herein, “difference in expression level”, and “modulation of expression level” and their synonyms, which are used interchangeably, refer to a significant difference in the expression of a gene. The terms encompass an increase in gene expression and/or decrease of gene expression.

The term “significant difference” in the context of the measured expression levels includes upregulation/over-expression/increase/induction and/or down-regulation/under-expression/decrease/reduction, or combinations thereof of examined genes (such as that a first gene of the examined expression profile may be upregulated whereas a second gene of the expression profile may be down-regulated).

In some embodiments, the determination of whether upregulation or down-regulation of a specific gene indicates the tested population is suitable for transplantation/administration is based on the specific genes expression level as described herein.

The terms “decrease”, “down-regulation”, and “reduction” are used interchangeably herein to refer to a statistically significant decrease in gene expression. In some embodiments, decrease refers to at least 1.2, at least 1.3, at least 1.4, at least 1.5, at least 1.6, at least 1.7, at least 1.8, at least 1.9, at least 2, at least 3, at least 4, at least 5, at least 6, at least 7, at least 8, at least 9, 10, 15, 25, 50, 75, 100, 200, 300, or any value therebetween, folds decrease. Each possibility represents a separate embodiment of the present invention.

As used herein, the terms “increase”, “upregulation”, and “induction” are used interchangeably herein to refer to a statistically significant increase in gene expression. In some embodiments, increase refers to at least 1.2, at least 1.3, at least 1.4, at least 1.5, at least 1.6, at least 1.7, at least 1.8, at least 1.9, at least 2, at least 3, at least 4, at least 5, at least 6, at least 7, at least 8, at least 9, 10, 15, 20, 25, 30, 50, 75, 100, or any value therebetween, fold increase. Each possibility represents a separate embodiment of the present invention.

In some embodiments, the invention makes use of differences in expression levels of target versus control cells.

In some embodiments, the target cell population is characterized by differences in expression levels of a plurality of genes as described herein.

According to some embodiments, there is provided a method for identifying target mesenchymal cells as being suitable for any one of: inhibiting an immune response in a subject in need thereof, treating or preventing acute respiratory distress syndrome, treating or preventing pulmonary manifestations of Covid-19, treating or preventing cytokine storm or cytokine release syndrome or any combination thereof, comprising determining the expression levels of one or more genes being selected from: BCL2, FGF7, HSP90AA1, MTOR, AKT1, PAK1, THBD, EDIL3, AMOT, and IL6R, in the target mesenchymal cells, wherein overexpression of BCL2 and FGF7 genes compared to expression of the BCL2 and the FGF7 in control mesenchymal cells; and under-expression of HSP90AA1, MTOR, and AKT1 genes compared to expression of the, HSP90AA1, the MTOR and AKT1 in control mesenchymal cells, is indicative of the target mesenchymal cells being suitable for any one of: inhibiting an immune response in a subject in need thereof, treating or preventing acute respiratory distress syndrome, treating or preventing pulmonary manifestations of Covid-19, treating or preventing cytokine storm or cytokine release syndrome or any combination thereof.

In some embodiments, target comprises being subjected to a combination of at least 2, 3, 4, 5, 6, 7, or 8 conditions selected from a group of conditions: (a) hypoxia, (b) serum-deprived, (c) oxidative stress, (d) hypothermia, (e) hyperthermia, (f) over confluency, (g) hydrostatic pressure, (h) dynamic or cyclic pressure, (i) shear forces, (j) agitation, (k) exposure to charged surfaces, (l) under confluency, or (m) any combination of (a) to (l).

### Determination of Gene Expression

Gene expression is the transcription of DNA into messenger RNA by RNA polymerase.

The term “expression” as used herein n refers to the biosynthesis of a gene product, including the transcription and/or translation of the gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Upregulation describes a gene that has been observed to have a higher expression (e.g., higher mRNA levels) in one sample (e.g., a sample suitable for transplantation) compared to another (e.g., a control sample). Down-regulation describes a gene that has been observed to have a lower expression (e.g., lower mRNA levels) in one sample (e.g., a sample suitable for transplantation) compared to another (e.g., a control sample).

In another embodiment, the gene expression is measured at the nucleic acid (mRNA, cDNA) level.

The term “nucleic acid” is well known in the art. A “nucleic acid” as used herein will generally refer to a molecule (i.e., a strand) of DNA, RNA, or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, a uracil “U” or a C). The terms “polynucleotide,” “polynucleotide sequence,” “nucleic acid sequence,” and “nucleic acid molecule” are used interchangeably herein.

Numerous detection and quantification technologies may be used to determine the expression level of the plurality of nucleic acids, including but not limited to: PCR, RT-PCR; RT-qPCR; NASBA; Northern blot technology; a hybridization array; branched nucleic acid amplification/technology; TMA; LCR; High-throughput sequencing or next-generation sequencing (NGS) methods such as RNA-seq, in situ hybridization technology; and amplification process followed by HPLC detection or MALDI-TOF mass spectrometry.

In embodiments of the invention, all or part of a nucleic acid may be amplified and detected by methods such as the polymerase chain reaction (PCR) and variations thereof, such as but not limited to reverse transcription PCR and real-time PCR (including as a means of measuring the initial amounts of mRNA copies for each sequence in a sample). Such methods would utilize one or two primers that are complementary to portions of a nucleic acid, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing sequences) under conditions that allow for their hybridization. Additional methods to detect the expression of expressed nucleic acids include RNase protection assays, including liquid phase hybridizations, and in situ hybridization of cells.

As would be understood by the skilled person, detection of expression of nucleic acids may be performed by the detection of expression of any appropriate portion or fragment of these nucleic acids or the entire nucleic acids. Preferably, the portions are sufficiently large to contain unique sequences relative to other sequences expressed in a sample. Moreover, the skilled person would recognize that either strand of a nucleic acid may be detected as an indicator of the expression of the nucleic acid. This follows because the nucleic acids are expressed as RNA molecules in cells, which may be converted to cDNA molecules for ease of manipulation and detection. The resultant cDNA molecules may have the sequences of the expressed RNA as well as those of the complementary strand thereto. Thus, either the RNA sequence strand or the complementary strand may be detected. Of course, it is also possible to detect the expressed RNA without conversion to cDNA.

In an embodiment, the method comprises performing a reverse transcription of mRNA molecules present in a sample; and amplifying the target cDNA and the one or more control cDNAs using primers hybridizing to the cDNAs.

A common technology used for measuring RNA abundance is RT-qPCR, where reverse transcription (RT) is followed by real-time quantitative PCR (qPCR). Commercially available systems for quantitative PCR may be used, for example, “Real-Time PCR System” of Applied Biosystems®, LightCycler® from Roche, iCycler® from BioRad®, and others. Reverse transcription first generates a DNA template from the RNA. This single-stranded template is called cDNA. The cDNA template is then amplified in the quantitative step, during which the fluorescence emitted by labeled hybridization probes or intercalating dyes changes as the DNA amplification process progresses. Quantitative PCR produces a measurement of an increase or decrease in copies of the original RNA and has been used to attempt to define changes in gene expression in cancer tissue as compared to comparable healthy tissues (Nolan T, et al. Nat Protoc 1:1559-1582, 2006; Paik S. The Oncologist 12:631-635, 2007; Costa C, et al. Transl Lung Cancer Research 2:87-91, 2013).

Massive parallel sequencing made possible by next-generation sequencing (NGS) technologies is another way to approach the enumeration of RNA transcripts in a tissue sample, and RNA-seq is a method that utilizes this. It is currently the most powerful analytical tool used for transcriptome analyses, including gene expression level differences between different physiological or culture conditions or changes that occur during development or over the course of disease progression. Specifically, RNA-seq can be used to study phenomena such as gene expression changes, alternative splicing events, allele-specific gene expression, and chimeric transcripts, including gene fusion events, novel transcripts, and RNA editing.

As used herein, the terms “amplification” or “amplify” mean one or more methods known in the art for copying a target nucleic acid, e.g., the genes listed in Table 1, thereby increasing the number of copies of a selected nucleic acid sequence. Amplification may be exponential or linear. In a particular embodiment, the target nucleic acid is RNA.

As used herein, “nucleic acid” refers broadly to segments of a chromosome, segments or portions of DNA, cDNA, and/or RNA. A nucleic acid may be derived or obtained from an originally isolated nucleic acid sample from any source (e.g., isolated from, purified from, amplified from, cloned from, or reverse transcribed from sample DNA or RNA).

As used herein, the term “oligonucleotide” refers to a short polymer composed of deoxyribonucleotides, ribonucleotides, or any combination thereof. Oligonucleotides are generally between about 10 and about 100 nucleotides in length. Oligonucleotides are typically 15 to 70 nucleotides long, with 20 to 26 nucleotides being the most common. An oligonucleotide may be used as a primer or as a probe. An oligonucleotide that is specific for a nucleic acid is one that, under the appropriate hybridization or washing conditions, is capable of hybridizing to the target of interest and not substantially hybridizing to nucleic acids that are not of interest. Higher levels of sequence identity are preferred and include at least 75%, at least 80%, at least 85%, at least 90%, or at least 95% sequence identity.

As used herein, a “fragment” in the context of a nucleic acid refers to a sequence of nucleotide residues which are at least about 5 nucleotides, at least about 7 nucleotides, at least about 9 nucleotides, at least about 11 nucleotides, or at least about 17 nucleotides. A fragment is typically less than about 300 nucleotides, less than about 100 nucleotides, less than about 75 nucleotides, less than about 50 nucleotides, or less than about 30 nucleotides. In certain embodiments, the fragments can be used in a polymerase chain reaction (PCR) or various hybridization procedures to identify or amplify identical or related DNA molecules.

As used herein, a “primer” for amplification is an oligonucleotide that specifically anneals to a target or marker nucleotide sequence. The 3′ nucleotide of the primer should be identical to the target or marker sequence at a corresponding nucleotide position for optimal primer extension by a polymerase. As used herein, a “forward primer” is a primer that anneals to the anti-sense strand of double-stranded DNA (dsDNA). A “reverse primer” anneals to the sense-strand of dsDNA.

As used herein, “target nucleic acid” refers to segments of a chromosome, a complete gene with or without intergenic sequence, segments or portions of a gene with or without intergenic sequence or sequence of nucleic acids to which probes or primers are designed. Target nucleic acids may be derived from genomic DNA, cDNA, or RNA. As used herein, target nucleic acid may be native DNA or a PCR-amplified product.

The detection methods described above are meant to exemplify how the present invention may be practiced and are not meant to limit the scope of the invention. It is contemplated that other sequence-based methodologies for detecting the presence of nucleic acid in a subject sample may be employed according to the invention.

In certain embodiments, one or more algorithms or computer programs may be used to compare the quantified expression levels of each gene in the test sample against a control sample. Alternatively, one or more instructions for manually performing the necessary steps by a human can be provided.

Algorithms for determining and comparing pattern analysis include, but are not limited to, principal component analysis, Fischer linear analysis, neural network algorithms, genetic algorithms, fuzzy logic pattern recognition, and the like. After the analysis is completed, the resulting information can, for example, be displayed on display, transmitted to a host computer, or stored on a storage device for subsequent retrieval.

As used herein, the term “mesenchymal cell”, “mesenchymal stem cell”, “mesenchymal stromal cells”, or “MSC” refers to a cell capable of giving rise to differentiated cells in multiple mesenchymal lineages, specifically to osteoblasts, adipocytes, and chondroblasts. As used herein, the term “mesenchymal cell” or “MSC” refers to mesenchymal stromal cells (MSC) and/or adipose stem cells (ASC).

Generally, mesenchymal cells also have one or more of the following properties: an ability to undergo asymmetric replication that is where the two daughter cells after division can have different phenotypes; extensive self-renewal capacity; and clonal regeneration of the tissue in which they exist, for example, the non-hematopoietic cells of bone marrow. “Progenitor cells” differ from stem cells in that they typically do not have extensive self-renewal capacity.

In some embodiments, the cell population is a heterogeneous cell population. In some embodiments, the heterogeneous cell population comprises at least 10% cells, at least 20% cells, at least 30% cells, at least 50% cells, at least 50% cells, at least 60% cells, at least 70% cells, at least 80% cells or at least 90% cells having the expression profile described herein.

As used herein, the term “human adipose stem cells (ASCs)” refers to a heterogeneous population of cells originating from the vascular stromal fraction (SVF) of fat or adipose tissues that can be used as an alternative cell source for many different cell therapies. As used herein, ASCs comprise heterogeneous population of cells comprising a plurality of: adipose-derived stem cells (ASC) (CD34− CD45− CD11b−, CD19, HLA-DR−, CD105+, CD73+, CD90+), mesenchymal cells, mesenchymal stem cells, vascular smooth muscle cells (Smooth muscle alpha-actin positive, Desmin positive, h-caldesmon positive, Smooth muscle myosin heavy chain positive), adipogenic, chondrogenic and osteogenic cells in any combination of osteoprogenitors, osteoblasts, osteocytes, chondroblasts, chondrocytes and osteoclasts, as well as endothelial progenitor cells (EPCs) (CD31+CD34+ CD45− CD144+ CD146+ CD102), hematopoietic progenitor cells (HPCs− CD34+) and mature ECs (CD31+CD34+ CD45− CD90− CD144+ CD146+ CD105+).

As used herein, the term “ex-vivo” or “in-vitro” refers to a process in which cells are removed from a living organism and are propagated outside the organism. As used herein, the term “in-vivo” refers to any process that occurs inside a living organism.

### General

The term “subject” as used herein refers to an animal, more particularly to non-human mammals and a human organism. Non-human animal subjects may also include prenatal forms of animals, such as embryos or fetuses. Non-limiting examples of non-human animals include: horses, cows, camels, goats, sheep, dogs, cats, non-human primates, mice, rats, rabbits, hamsters, guinea pigs, and pigs. In one embodiment, the subject is a human. Human subjects may also include fetuses. In one embodiment, a subject in need thereof is a subject afflicted with and/or at risk of being afflicted with a condition associated with an increased immune response.

As used herein, the terms “treatment” or “treating” of a disease, disorder, or condition encompasses alleviation or prevention of at least one symptom thereof, a reduction in the severity thereof, or inhibition of the progression thereof. Treatment need not mean that the disease, disorder, or condition is totally cured. To be an effective treatment, a useful composition herein needs only to reduce the severity or the expected severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described target MSCs prior to the induction or onset of the disease/disorder process. This could be done where an individual has a genetic pedigree indicating a predisposition toward the occurrence of the disease/disorder to be prevented or when an individual is planned to undergo a procedure known to cause a condition to be prevented. For example, this might be true of an individual who is undergoing immunotherapy. The term “suppression” is used to describe a condition wherein the disease/disorder process has already begun, but obvious symptoms of the condition have yet to be realized. Thus, the individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, the term “condition” includes anatomic and physiological deviations from the normal that constitute an impairment of the normal state of the living animal or one of its parts that interrupts or modifies the performance of the bodily functions.

Any concentration ranges, percentage ranges, or ratio ranges recited herein are to be understood to include concentrations, percentages, or ratios of any integer within that range and fractions thereof, such as one-tenth and one-hundredth of an integer unless otherwise indicated.

Any number ranges recited herein relating to any physical feature, such as polymer subunits, size, or thickness, are to be understood to include any integer within the recited ranges unless otherwise indicated.

As used herein, the terms “subject” or “individual” or “animal” or “patient” or “mammal” refer to any subject, particularly a mammalian subject, for whom therapy is desired, for example, a human.

In the discussion, unless otherwise stated, adjectives such as “substantially” and “about” modifying a condition or relationship characteristic of a feature or features of an embodiment of the invention are understood to mean that the condition or characteristic is defined within tolerances that are acceptable for the operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an”, and “at least one” are used interchangeably in this application.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

For purposes of better understanding the present teachings and in no way limiting the scope of the teachings, unless otherwise indicated, all numbers expressing quantities, percentages or proportions, and other numerical values used in the specification and claims are to be understood as being target in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, “have”, and conjugates thereof are used to indicate that the object or objects of the verb are not necessarily a complete listing of components, elements or parts of the subject or subjects of the verb. Other terms as used herein are meant to be defined by their well-known meanings in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements.

Additional objects, advantages, and novel features of the present invention will become apparent to one ordinarily skilled in the art upon examination of the following examples, which are not intended to be limiting. Additionally, each of the various embodiments and aspects of the present invention, as delineated herein above and as claimed in the claims section below, finds experimental support in the following examples.

### EXAMPLES

Generally, the nomenclature used herein and the laboratory procedures utilized in the present invention include molecular, biochemical, bioengineering, bioprocessing, microbiological, and recombinant DNA techniques. Such techniques are thoroughly explained in the literature. See, for example, “Molecular Cloning: A Laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); Molecular Cell Biology Berk A. et al. 8th edition; Molecular Biotechnology: Principles and Applications of Recombinant DN, Glick BR. 5th edition; Culture of Animal Cells: A Manual of Basic Technique and Specialized Applications Freshney IR, 7th edition; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference. Other general references are provided throughout this document.

### Example 1

### Target MSCs are Characterized by a Particular Gene Expression Profile

The profile of differentially expressed genes (DEGs) in three batches (N=3) of target MSCs, such as ASCs, obtained from different donors compared to three paired batches of control MSCs, such as ASCs obtained from the same donors was assessed by RNA sequencing as briefly described below.

Total RNA was extracted from six samples using the RNeasy kit (Cat No. 74106). Frozen cells were disrupted in 600 μl buffer RLT and homogenized by needle and syringe. Quality control for total RNA was performed using the TapeStation 4200 (Agilent) with the RNA kit (Cat No. 5067-5576). The RINe value of all samples was 10, indicating excellent quality. Six RNAseq libraries were constructed simultaneously according to the manufacturer's protocol (NEBNext Ultra II Directional RNA Library Prep Kit for Illumina, Cat No. E7760) using 800 ng total RNA as starting material. mRNAs pull-down was performed using a Magnetic Isolation Module (NEB, cat no. E7490). After construction, each library's concentration was measured using Qubit (Invitrogen), and the size was determined using the TapeStation 4200 with the High Sensitivity D1000 kit (Cat No. 5067-5584). All libraries were mixed into a single tube with equal molarity. The RNAseq data was generated on Illumina NextSeq500, 150 cycles (75 paired-end), and Mid-output mode (Illumina, cat no. 20024904). Quality control was assessed using Fastqc (v0.11.5). Reads were trimmed for adapters, low quality 3′, and a minimum length of 20 using CUTADAPT (v1.12). Seventy-five bp paired-end reads were aligned to human reference genome (Homo_sapiens.GRCh38.dna.primary_assembly.fa downloaded from ENSEMBL) and annotation file (Homo_sapiens.GRCh38.92.gtf downloaded from ENSEMBL) using STAR aligner (v2.6.0a). The number of reads per gene was counted using Htseq (v0.9.1). Statistical analysis was performed using the DESeq2 R package (version 1.26.0, Genome Biology 2014 15:550).

The similarity between the samples of the target and control cells was evaluated using the DESeq2 R package producing a list of DEGs. Several genes that are known to be associated with the therapeutic potency of MSCs, were selected from this list and are presented in Table 1. These genes are presented along with their base-2 logarithm of the average fold-change (FC) in mRNA expression between target and control cells (Log₂FC), plus or minus the standard error (+SE) of the FC. The base-10 logarithm of the adjusted p-value for these DEGs and the FC thresholds measured in the data set are listed as well (the threshold for the gene THBD is calculated differently, as explained below).

TABLE 1
Selected DEGs in target MSCs

| | | | | Log₁₀ | |
|---|---|---|---|---|---|
| Gene Symbol | Gene Description | Log₂FC ± SE | | (adjusted p-value) | FC threshold |
| BCL2 | BCL2 apoptosis | 1.32 | ± 0.30 | −4.46 | ×1.55 |
| | regulator | | | | |
| FGF7 | fibroblast growth | 3.02 | ± +0.18 | −58.95 | ×6.28 |
| | factor 7 | | | | |
| HSP90AA1 | heat shock protein | −2.09 | ± 0.08 | −136.60 | ÷4.07 |
| | 90 alpha family class | | | | |
| | A member 1 | | | | |
| MTOR | mechanistic target of | −0.86 | ± 0.11 | −13.31 | 1.66 |
| | rapamycin kinase | | | | |
| AKT1 | AKT serine/threonine | −0.65 | ± 0.09 | −10.93 | 1.49 |
| | kinase 1 | | | | |
| PAK1 | p21 (RAC1) | −0.85 | ± 0.13 | −9.34 | 1.64 |
| | activated kinase 1 | | | | |
| THBD | thrombomodulin | 0.60 | ± 0.49 | −0.48 (ns) | 1.28 ^{(a)} |
| EDIL3 | EGF like repeats and | 3.03 | ± 0.17 | −70.98 | ×7.35 |
| | discoidin domains 3 | | | | |
| AMOT | angiomotin | 5.23 | ± 0.90 | −7.45 | ×1.55 |
| IL6R | interleukin 6 | 5.40 | ± 1.24 | −4.25 | ×10.55 |
| | receptor | | | | |

^{(a)} The FC threshold for the maximal reduced expression of THBD by target cells is evaluated based on the 95% confidence interval of the mean FC
according to the following calculation: 1.28 = 1/2^{0.60-19.96×0.49}

Table 1 shows, for example, that target MSCs express significantly higher levels of the genes BCL2 and FGF7 by an average FC of 2^{1.32±0.30} (p=10^{-4.46}) and 2^{3.02±0.18} (p=10^{-58.95}), respectively, or by at least 1.55-fold and a 6.28-fold increase compared to control MSCs. Similarly, Table 1 shows that target MSCs express significantly lower levels of the genes HSP90AA1, MTOR, and AKT1 by an average FC of 2^{-2.09±0.08} (p=10^{-136.60}), 2^{-0.86±0.11} (p=10^{-13.31}), and 2^{-0.65±0.09} (p=10^{-10.93}), respectively, or by at least 4.07-fold, 1.66-fold, and a 1.49-fold decrease compared to control MSCs.

In addition, Table 1 shows that target MSCs, such as ASCs, express a significantly lower level of the gene PAK1 by at least 1.64-fold (p=10-9.34) compared to control MSCs. The average level of the gene THBD measured across three target MSCs, batches is not significantly (ns) different compared to the average levels of THBD measured across the three paired batches of control MSCs (p=10-0.48). Since THBD does not change significantly, the threshold for its change was not determined based on a specific data point, as the inventors have done for other genes. Alternatively, the threshold for THBD was determined based on the 95% confidence interval of the mean FC, as exemplified in Comment (a) for Table 1. Also, Table 1 shows that target MSCs, express significantly higher levels of the genes EDIL3 and AMOT by at least 7.35-fold (p=10^{-70.98}) and 1.55-fold (p=10^{-7.45}) respectively, compared to control MSCs. Lastly, the gene IL6R was expressed by all three batches of target MSCs, but only one of the three paired batches of control MSCs. This one batch of control MSCs that did express IL6R presented a very low level of this gene; precisely, 3 reads only were measured in the control, compared to 23 to 37 reads in the target MSC batches. This low level of IL6R gene expression in just one of the three control batches points to a possible measurement artifact in this batch. Collectively the results obtained regarding the IL6R gene imply that this gene is either expressed de novo following or over-expressed by at least 10.55-fold in target MSCs of the invention, compared to control cells.

### Example 2

### Target Mesenchymal Stromal Cells Present Increased Cell Granularity

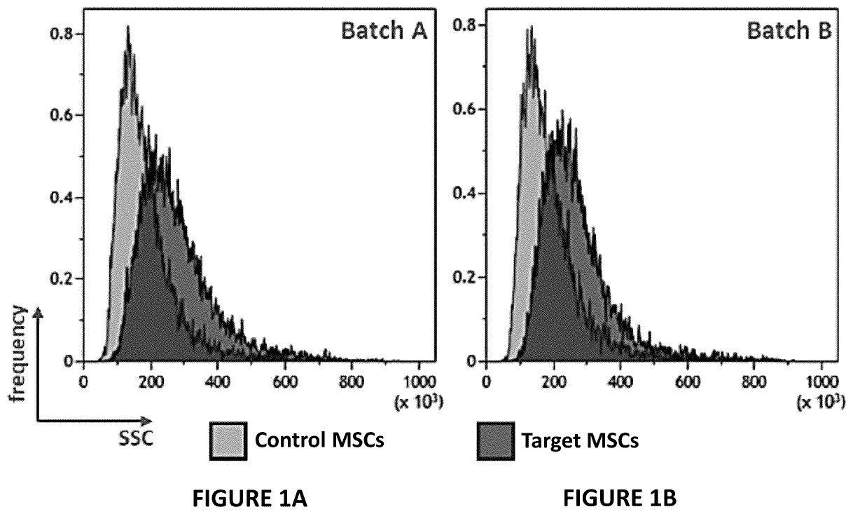

A flow cytometric analysis of two representative batches of target MSCs, such as ASCs, and their paired control MSCs revealed a substantial increase of at least 40% in the geometric mean of the side scatter (SSC) parameter measured for target MSCs, as shown in FIG. 1, pointing to their increased cell granularity compared to control.

### Example 3

Target Mesenchymal Stromal Cells are Immunosuppressive and have an Enhanced Ability to Inhibit the Proliferation of T Cells

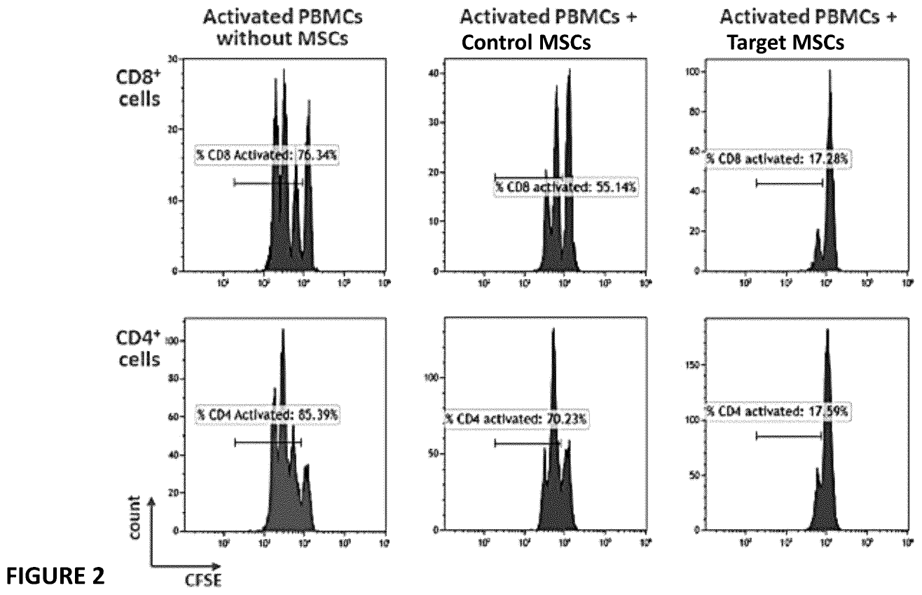

An assay was developed to compare the immunosuppressive potency of target and control MSCs as their ability to inhibit T cells' proliferation following immune activation by exposure to non-specific stimuli, otherwise leading to extensive proliferation. The assay is based on a standard proliferation assay for peripheral blood mononuclear cells (PBMCs) pre-labeled with a fluorescent dye carboxyfluorescein succinimidyl ester (CFSE). This dye is associated with the cells' membranes and allows for tracking their proliferation. Following their non-specific activation by exposure to anti-CD3 and anti-CD28 antibodies, the PBMCs start proliferating. This proliferation results in dilution of the membrane-associated dye between the progeny cells and a subsequent two-fold reduction in the daughter cells' fluorescent intensity with each cell doubling. When analyzed by flow cytometry, this dilution results in a typical multi-modal histogram, as shown in FIG. 2, for activated CD8⁺ and CD4⁻ PBMCs without MSCs, with generation zero (G0) cells presenting the highest fluorescent intensity that is reduced in subsequent generations G1, G2, and G3. By co-labeling the PBMCs with fluorescent anti-CD8 or anti-CD4 antibodies, this CFSE analysis allows determining the percentage of CD8⁺ and CD4⁺ T cells in generations other than G0 as a measure of their immune activation. Using this experimental setup, it is possible to co-culture pre-labeled and immune-activated PBMCs with target and control MSCs and assess their effect on the T cells' proliferation and activation as a measure of their immunosuppressive capacity.

In FIG. 2, the effect of target and control MSCs on the proliferation of immune-activated T cells was recorded. Target and control MSCs were seeded in a 96-well plate at a concentration of 20 thousand cells/well. To control the number of reacting MSCs, their proliferation was halted by treatment with Mitomycin C. After removing the Mitomycin C, 200 thousand PBMCs, pre-stained with CFSE, were added into each well containing MSCs, as well as an additional control well without MSCs. The T cells in the PBMCs are then activated by adding T Cell TransAct beads (Miltenyi Biotec) into the wells conjugated with anti-CD3 anti-CD28 antibodies. Following 76 hours of co-culture, the suspension cells, consisting mainly of PBMCs, are removed from the wells, co-stained with anti-CD4 and anti-CD8 antibodies, and analyzed by flow cytometry for CFSE labeling against appropriate controls. The percent of activated CD8⁺ or CD4+ T cells is then calculated for each well as the ratio between the number of proliferative T cells—i.e., cells found in generations other than zero—and the total number of T cells analyzed (generation zero included).

As shown in FIG. 2, control MSCs have managed to reduce the percentage of activated CD8⁺ T cells from 76% to 55% and from 85% to 70% for CD4+ T cells. The effect of the target MSCs was much stronger, reducing the percentage of activated CD8⁺ and CD4+ T cells to as low as 17% or 18%, respectively, pointing to the improved immunosuppressive potency of the target MSCs compared to the control ones.

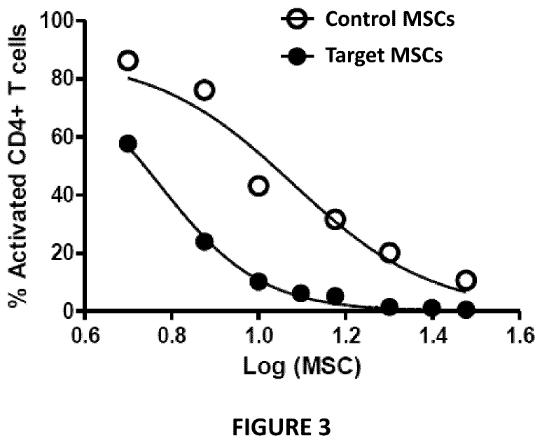

To measure the inhibitory effect of the target and control MSCs on the proliferation of immune-activated T cells, MSCs were seeded in different concentrations, up to 30 thousand cells per well in a 96-well plate. Following overnight incubation of the MSCs and proliferation arrest with mitomycin C, as described in the example above, CFSE-stained PBMCs were added to the target and control MSCs and immune activated. Four days after T cell activation, the MSCs' immunomodulatory effect was assessed by flow cytometry analysis of the activated CD4+ T cells and by calculating the inhibitory effect (as inhibitory concentration 50% or IC50) of increasing MSCs numbers on the percentage of activated CD4+ T cells. The IC50 calculation was performed using the four-parameters logistic regression model (4PL). Maximal activation was set according to the activated PBMCs control group without MSCs. As shown in FIG. 3, target MSCs are characterized by a dramatic improvement of T cell proliferation inhibition, and reduction of IC50 compared to control MSCs.

In FIG. 3, the effect of different concentrations of target and control MSCs on the proliferation of immune-activated T cells was recorded. Target and control MSCs were seeded in a 96-well plate at different concentrations of up to 30 thousand cells/well. To control the number of reacting MSCs, their proliferation was halted by treatment with Mitomycin C. After removing the Mitomycin C, 200 thousand PBMCs, pre-stained with CFSE, were added into each well containing MSCs, as well as additional control wells without MSCs. The T cells in the PBMCs are then activated by adding T Cell TransAct beads (Miltenyi Biotec) into the wells conjugated with anti-CD3 anti-CD28 antibodies. Following four days of co-culture, the suspension cells, consisting mainly of PBMCs, are removed from the wells, co-stained with anti-CD4, and analyzed by flow cytometry for CFSE labeling against appropriate controls. The percent of activated CD4⁺ T cells is then calculated for each well and plotted against the log₁₀ of the number of MSCs seeded in thousands (for example, x=1 denotes 10,000 cells/well). To calculate the IC50 values, a four-parameter logistic (4PL) regression curve was then fitted onto the data with high correlation (R²=0.9472 for control MSCs and R²=0.9953 for target MSCs).

A robust immunomodulatory capacity was further demonstrated in vitro by the target MSCs' disclosed herein that inhibited neutrophils' reactive oxygen species (ROS) production by up to 80% within an hour following activation (IC50 19K MSC/200K neutrophils).

### Example 4

### Target Mesenchymal Stromal Cells Induce an Anti-Inflammatory Effect Reducing Lung Edema in an In Vivo Murine Model for Acute Lung Inflammation

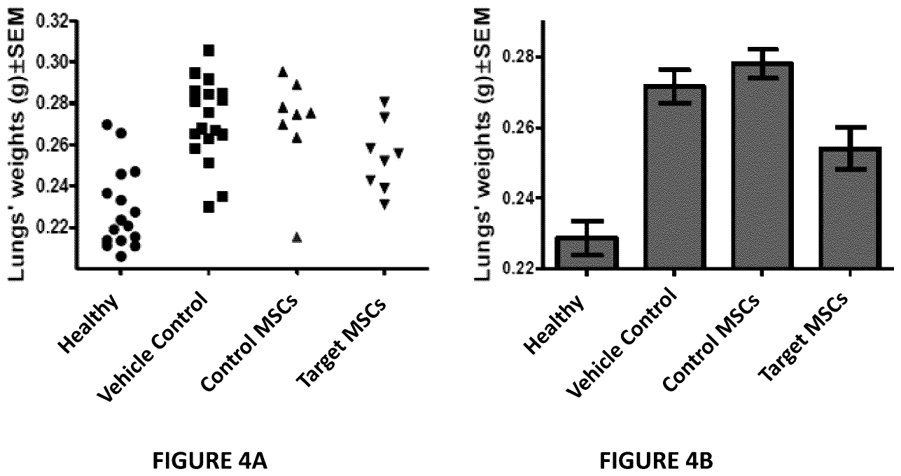

An acute lung injury (ALI) model was established in immunocompetent mice by intratracheal injection of lipopolysaccharide (LPS) to study the anti-inflammatory effect of target MSCs as disclosed herein. This model is widely accepted for inducing lung inflammation characterized by excessive accumulation of leukocytes and fluids in the lungs leading to edema. Lung edema can be directly evaluated in animal models by measuring the lungs' weights and assessing the excess fluid accumulation and inflammation. As shown in FIG. 4, target MSCs, but not control MSCs, reduced the lungs' weights, pointing to their enhanced anti-inflammatory potency.

In FIG. 4, the effect of target and control MSCs on lungs' weights in ALI model animals was recorded. Target MSCs were injected IV 6 hours post-induction of an acute lung injury (ALI) model in mice. Animals were sacrificed 18 hours post-treatment, and their lungs were harvested, weighed, and compared to lungs harvested from healthy untreated animals as well as model animals injected with Vehicle Control and control MSCs.

### Example 5

### Target Mesenchymal Stromal Cells Reduce Lung Infiltration of Lymphocytes, Monocytes, and Neutrophils in an In Vivo Murine Model for Acute Lung Inflammation

To assess the effect of target MSCs on the different arms of the immune response, an acute lung injury (ALI) model was established in immunocompetent mice by intratracheal injection of lipopolysaccharide (LPS). This model is widely accepted for inducing lung inflammation characterized by excessive accumulation of leukocytes of both immune arms in the lungs. The target MSCs were injected IV six hours post LPS injection, and animals were sacrificed 24 hours post LPS injection (18 hours post-intervention). Control groups included healthy animals and model animals injected with vehicle control. Bronchoalveolar lavage fluid (BALF) was then extracted from the animals' lungs by washes with PBS and concentrated by centrifugation. BALF cells from every two animals belonging to the same group were pooled and analyzed by complete blood count (CBC) protocol.

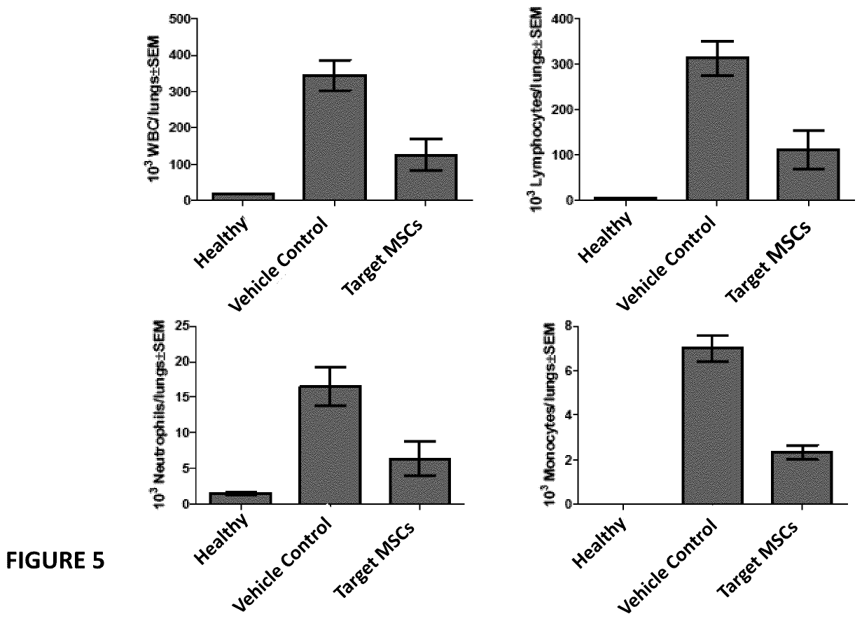

In FIG. 5, immune cell counts in the BALF of ALI model animals post-treatment with target MSCs, are provided. Specifically, target MSCs were injected 6 hours post-ALI model induction, and animals were sacrificed 18 hours post-treatment. The animals' BALF cells were harvested, concentrated, pooled for every two animals, and subjected to CBC compared to BALF cells harvested from healthy untreated animals and model animals injected with the vehicle control item. Results are presented as the average counts±SEM of the total white blood cells (WBCs), lymphocytes, neutrophils, and monocytes.

As further shown in FIG. 5, ALI induction led to a dramatic increase in the level of all immune cells measured in the BALF of the Vehicle Control animals relative to healthy animals. Specifically, an increase was measured in the levels of the total WBCs (p<0.001) and of the lymphocytes, wherein the latter belong to the adaptive immune arm (p<0.001), as well as neutrophils (p<0.01) and monocytes (no p-value due to zero values in the non-treated group) that belong to the innate immune arm. Relative to the Vehicle Control group, target MSCs have lowered the levels of all immune cell counts by at least 40%, pointing to their inhibitory effect on immune cells of both the adaptive and innate immune arms.

### Example 6

### Soluble Secretion of Target Mesenchymal Stromal Cells Promotes Migration of Endothelial and Epithelial Cells and Inhibits Fibroblastic Cells Migration in Tissue Cultures

To assess the effect of target MSCs, such as ASCs on tissue regeneration, conditioned media (CM) was collected from these cells containing soluble secreted factors. The CM was concentrated 50 to 100-fold by ultrafiltration via 10 kDa filters. Confluent tissue cultures of endothelial, epithelial, and fibroblastic cells were scratched to create a culture wound or gap, washed, and added with CM to a final concentration of 5 mg/ml in the cells' recommended medium without serum. Wounded cultures without CM and with or without serum served as negative and positive controls, respectively. Cultures were imaged shortly after their scratching (0 hours) and 20 to 24 hours thereafter to assess cell migration and wound healing.

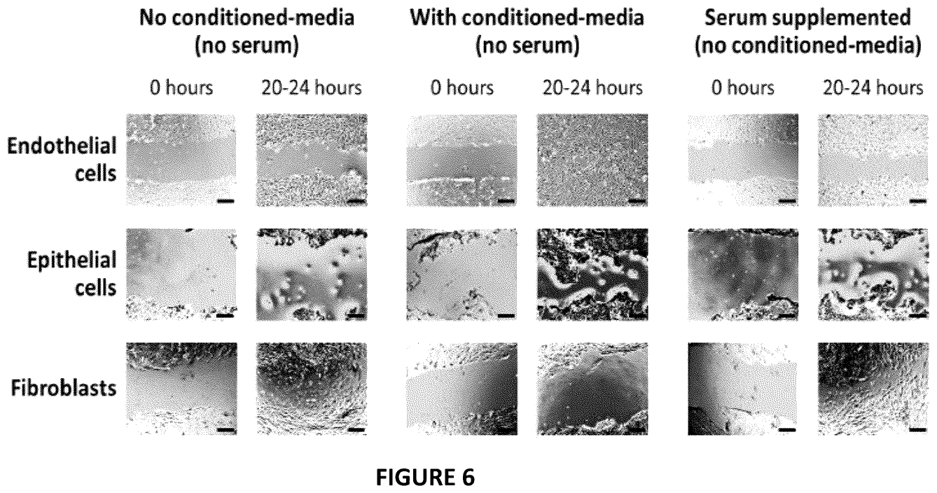

As shown in FIG. 6, wounds in endothelial and epithelial cell cultures were closed more rapidly in the presence of CM than without CM or in serum presence. In contrast, fibroblasts' wounds closed slower in the presence of CM compared to both controls. These results show that soluble factors secreted by target MSC encourage the migration of endothelial and epithelial cells and inhibit the migration of fibroblastic cells. Collectively, these results point to the ability of target MSCs to promote tissue regeneration and wound healing by inducing re-reendothelialization and re-epithelialization and possibly inhibiting fibrosis.

### Example 7

### Target Mesenchymal Stromal Cells Therapy for Acute Respiratory Distress Syndrome (ARDS)

The inventors initiated a multi-center Phase II trial in severe COVID-19 patients that was recently concluded.

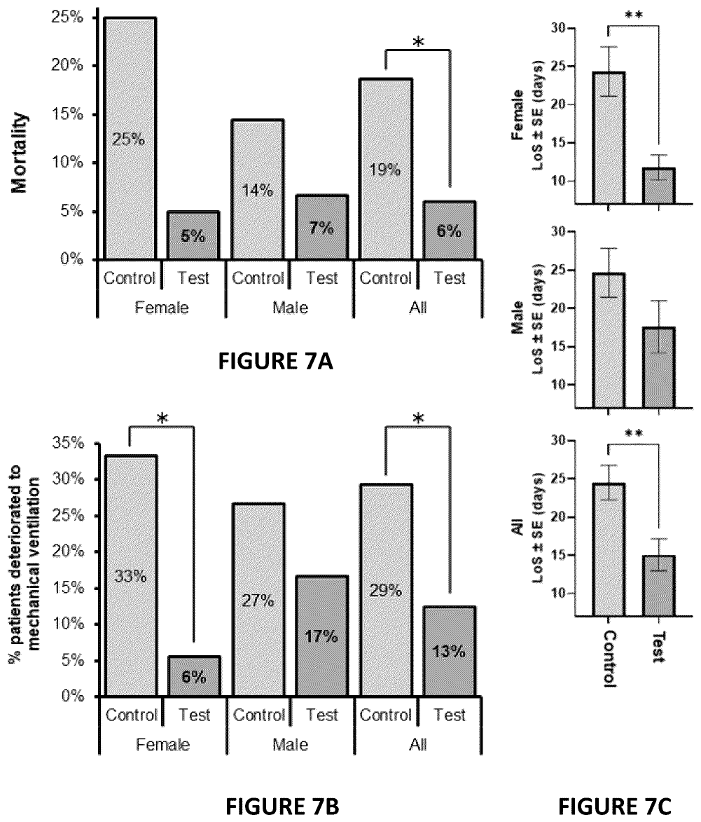

The Phase II trial included 50 severe Covid-19 patients suffering from diffuse pneumonia and oxygen desaturation treated with up to 3 doses (1.5×10⁶ cells/kg on days 1, 3, and 5) of the target MSCs disclosed herein, on top of the Standard of Care (SoC), and 150 similar severe control patients treated by the SoC only and stratified according to gender, age, and comorbidities. A substantial 68% reduction in the mortality rate of the test patients was measured (FIG. 7A, p<0.05), along with a 57% drop in their risk of intubation relative to the control (FIG. 7B, p<0.05). Over 50% of the patients treated with the target MSCs disclosed herein were released from the hospital within two days after treatment, and a 38% reduction was measured in the hospital length of stay (LoS) of patients having LoS>7 days (FIG. 7C, p<0.01).

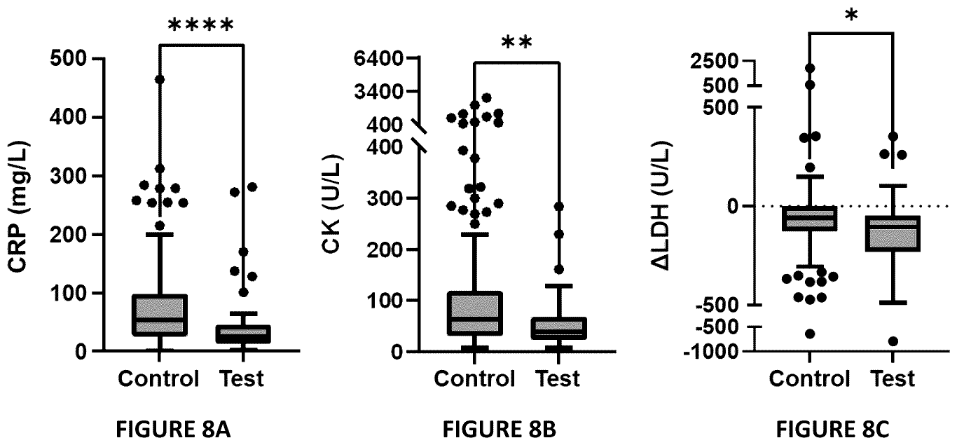

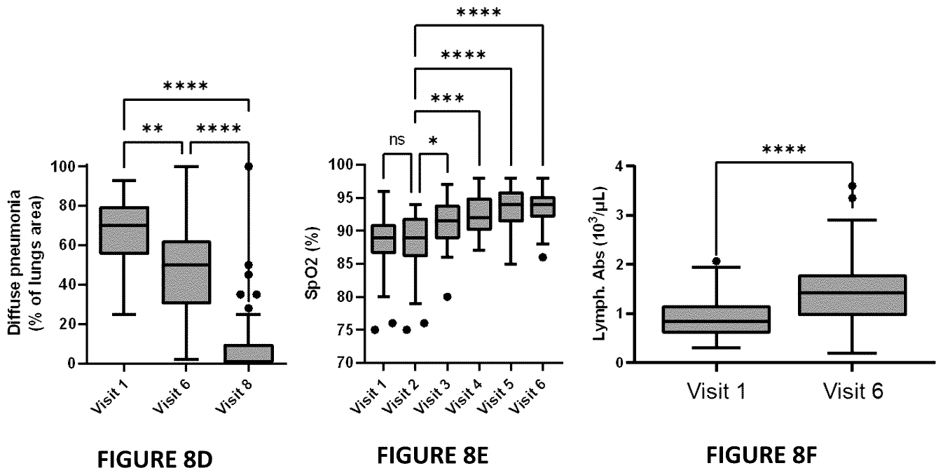

Starting from a similar baseline as the control, the median CRP (FIG. 8A) and CK levels (FIG. 8B) of the test patients, after the target MSCs treatment, ended at 52% (p<0.0001) and 33% (p<0.01) lower than their respective control levels. More significant reduction (p<0.05) was also measured in the median LDH level of test patients administered with target MSCs compared to control patients (FIG. 8C). As further shown in FIGS. 8D-8F, the more profound improvements in inflammatory and tissue damage markers observed in test patients were accompanied by a rapid recovery in pneumonia, respiratory functions, and lymphopenia, emphasizing the powerful effect of the target MSCs disclosed herein. In conclusion, the inventors show that the target MSCs disclosed herein save patients' lives and accelerate their healing, possibly reducing the risk of long-term damages while freeing ICU beds allowing better care for other patients, and reducing the burden associated with hospitalization and additional long-term healthcare costs.

### Example 8

### Target Mesenchymal Stromal Cells Therapy for Immunotherapy-Related Cytokine Release Syndrome

Cytokine Release Syndrome (CRS) and Immune effector Cell-Associated Neurotoxicity Syndrome (ICANS) are related side effects of immunotherapies seen in up to 76% of patients treated with chimeric antigen receptor T cell therapy (CAR-T) and 48% of those treated with bispecific T-cell engagers (BiTEs). In up to 27% of the patients, these syndromes may lead to severe consequences. Current treatments for severe CRS are ineffective in >30% of the cases and can worsen ICANS prognosis, calling for novel treatments, especially in light of the expanding use of immunotherapies.

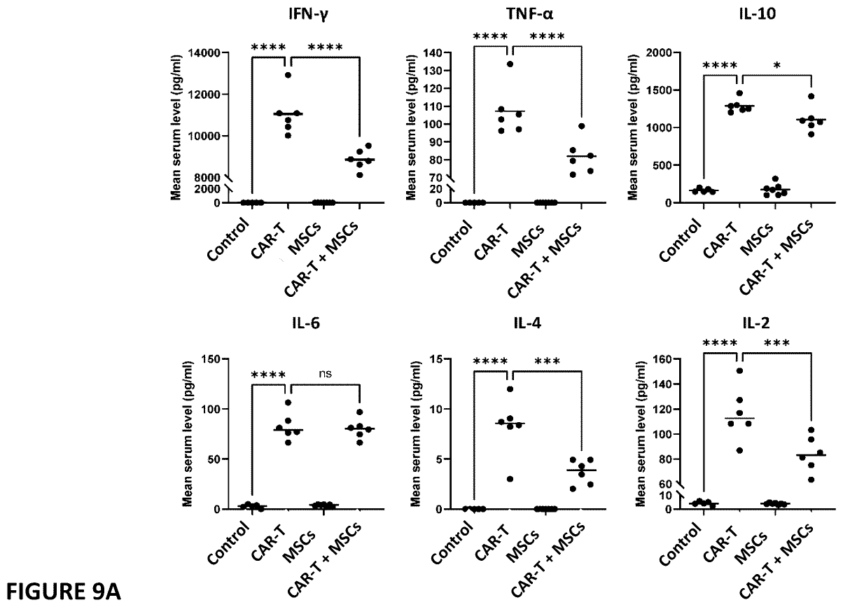

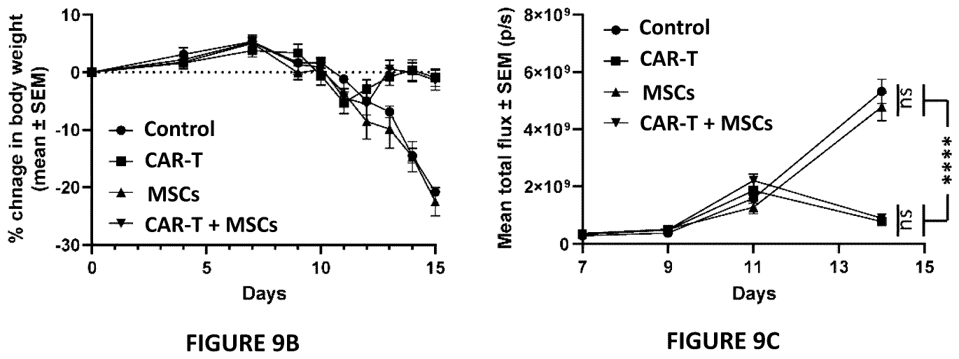

A highly translational and validated CRS model was established in humanized NSG mice bearing human PBMCs, B-cell lymphoma, and CAR-T cells. CAR-T introduction significantly increased the serum levels of proinflammatory cytokines in model animals, indicative of CRS (FIG. 9A). Two intravenous (IV) injections of the target MSCs disclosed herein were well-tolerated in this model (FIG. 9B) and did not obstruct the CAR-Ts' ability to inhibit tumor growth by 89% (FIG. 9C, p<0.0001). Remarkably, significant reductions in all pro-inflammatory cytokines tested (excluding IL-6) were measured in model animals treated with the target MSCs disclosed herein, substantiating their potential to treat CRS (FIG. 9A).

### Example 9

### Target Mesenchymal Stromal Cells Express Higher Levels of Immunomodulatory IDO1 Compared to Control Cells Upon Exposure to Inflammatory Factors

Indoleamine 2, 3-dioxygenase (IDO1) is the first and rate-limiting catabolic enzyme in the degradation pathway of the essential amino acid tryptophan. By cleaving the aromatic indole ring of tryptophan, IDO initiates the production of a variety of tryptophan degradation products called “kynurenines” that are known to exert essential immunoregulatory functions. Through IDO degradation of tryptophan, cells that express the enzyme mediate potent effects on metabolic events responsible for innate and adaptive immune responses to inflammatory insults. In addition, IDO1 was shown to alter immune responses through various mechanisms depending on the regulation of cell metabolism.

MSCs can express IDO1 following stimulation by the pro-inflammatory cytokines interferon-γ (IFNγ). In humans, MSCs respond to pro-inflammatory cytokines in part by IDO1 secretion, which suppresses this inflammatory response.

An experiment was devised by the inventors to compare the immunomodulatory capacity of target and control MSCs in terms of the level of IDO1 mRNA production following exposure to the inflammatory cytokine IFNγ. Briefly, target and control MSCs were seeded in 6-well plates (1.5·10⁶ cells/well) and added with IFNγ (50 ng/ml). After four hours of incubation, the cells are harvested, and their RNA is extracted and measured for the IDO1 expression (normalized to housekeeping gene RPLP0) using RT-qPCR.

Analysis of six independent batches revealed an average increase of 6.8-fold (±SE of 2.3-fold) in IDO1 expression by target MSCs compared to control MSCs.

All publications, patents, and patent applications mentioned in this specification are herein incorporated in their entirety by references into the specification, to the same extent as if each individual publication, patent, or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A composition comprising target mesenchymal cells characterized by overexpression of BCL2 and FGF7 genes compared to expression of said BCL2 and said FGF7 in control mesenchymal cells; and under-expression of HSP90AA1, MTOR, and AKT1 genes compared to expression of said, HSP90AA1, said MTOR and said AKT1 in said control mesenchymal cells.

2. The composition of claim 1, wherein said target mesenchymal cells are further characterized by under-expression of PAK1 gene compared to expression of said PAK1 gene in said control mesenchymal cells and less than 22% under-expression of THBD gene compared to expression of said THBD gene in said control mesenchymal cells.

3. The composition of claim 1, wherein said target mesenchymal cells are further characterized by overexpression of EDIL3 gene, AMOT gene, or their combination, compared to expression of said EDIL3, AMOT, or both in said control mesenchymal cells.

4. The composition of claim 1, wherein said target mesenchymal cells are further characterized by overexpression or de novo expression of the IL6R gene compared to expression of said IL6R in said control mesenchymal cells.

5. The composition of claim 1, wherein said target mesenchymal cells are further characterized by increased cell granularity compared to control mesenchymal cells, wherein said target mesenchymal cells are further characterized by a larger proportion of cells in the G1 phase of a cell cycle compared to control mesenchymal cells, or a combination thereof.

6. The composition of claim 1, wherein said overexpression of BCL2 is by at least 1.5-fold increase, wherein said overexpression of FGF7 is by at least 5-fold increase, wherein said under-expression of HSP90AA1 is by at least 2-fold decrease, wherein said under-expression of MTOR is by at least 1.5-fold decrease, wherein said under-expression of AKT1 is by at least 1.5-fold decrease, wherein said under-expression of PAK1 is by at least 1.4-fold decrease, or any combination thereof.

7. The composition of claim 2, wherein said under-expression of PAK1 is by at least a 1.8-fold decrease.

8. The composition of claim 3, wherein said overexpression of EDIL3 is by at least 7.3-fold increase, wherein said overexpression of AMOT is by at least 1.5-fold increase, or both.

9. The composition of claim 1, wherein said mesenchymal cells are derived from: adipose tissue, umbilical cord, chorionic placenta, bone marrow, amniotic placenta, dental pulp, amniotic fluid, peripheral blood, synovium, synovial fluid, endometrium, skin, muscle, embryonic stem cells, induced pluripotent stem cells, or any combination thereof.

10. The composition of claim 1, wherein said target mesenchymal cells are modified by subjecting the cells to a combination of conditions selected from the group consisting of: (a) hypoxia, (b) starvation, (c) oxidative stress, (d) hypothermia, (e) hyperthermia, (f) over confluency, (g) hydrostatic pressure, (h) dynamic or cyclic pressure, (i) shear forces, (j) agitation, (k) exposure to charged surfaces, (l) under confluency; and (m) any combination of (a) to (l).

11. (canceled)

12. A method for inhibiting an immune response in a subject in need thereof, comprising administering to said subject a therapeutically effective amount of the composition of claim 1, thereby inhibiting an immune response in a subject in need thereof.

13. The method of claim 11, further comprising reducing inflammation in said subject.

14. The method of claim 12, wherein said immune response is an adaptive immune response, an innate immune response, or both.

15. The method of claim 12, wherein said subject is: afflicted with a cytokine storm, afflicted with a cytokine release syndrome, afflicted with SEPSIS and/or SIRS, at risk of developing a cytokine storm, at risk of developing a cytokine release syndrome, or at risk of developing SEPSIS and/or SIRS.

16. The method of claim 12, wherein said inhibiting comprises inhibiting the activity of CD4+ cells, CD8+ cells, or both.

17. The method of claim 12, wherein said inhibiting comprises inhibiting cytokine production, secretion, or both, in said subject.

18. A method for inducing tissue regeneration in an injured tissue or a degenerated tissue in a subject in need thereof, comprising administering to said subject a therapeutically effective amount of the composition of claim 1, thereby inducing tissue regeneration in an injured tissue or a degenerated tissue in a subject in need thereof.

19. The method of claim 18, wherein said tissue regeneration comprises induction of wound healing, reendothelization, reepithelization, cell proliferation, cell migration, or any combination thereof.

20. The method of claim 18, wherein said tissue regeneration comprises inhibiting or controlling tissue fibrosis.
